(19) United States
(12) Patent Application Publication
(10) Pub. No.: US 2007/0161001 A1
(43) Pub. Date: Jul. 12, 2007

(54) Quantifying and profiling antibody and t cell receptor gene expression

(76) Inventor: Dena Leshkowitz, Yahud (IL)

Correspondence Address: Martin D Moynihan;Prtsi Inc, P O Box 16446, Arlington, VA, 22215, US

(21) Appl. No.: 10/591,442

(22) PCT Filed: Mar. 6, 2005
(86) PCT No.: PCT/IL05/00263

§ 371 Date: Sep. 1, 2006

Related U.S. Application Data
(60) Provisional application No. 60/549,541, filed on Mar. 4, 2004.

(51) Int. Cl. C12Q 1/68; G06F 19/00

(52) U.S. Cl. 435/6; 702/20

(57) ABSTRACT

A method of sequencing a population of polynucleotides encoding antibodies or T-cell receptors. Also provided are a method of quantifying an expression of a population of polynucleotides encoding antibodies or T-cell receptors as well as an oligonucleotide library for sequencing by hybridization of polynucleotides encoding variable regions of antibodies or T cell receptors.

### FIELD OF THE INVENTION

The present invention relates to the sequencing of expressed genes belonging to the immunoglobulin gene superfamily, particularly immunoglobulins and T cell receptors. In particular, the present invention provides methods of sequencing expressed genes in a non-clonal population of cells of the immune system, generating profiles of the genes expressed, and correlating the data generated with states of disease or health.

### BACKGROUND OF THE INVENTION

Diseases associated with a protective or pathogenic antigen specific immune response, such as infectious, autoimmune, allergic, transplantation related, malignant, and inflammatory diseases, include numerous highly debilitating and/or lethal diseases whose medical management is suboptimal, for example, with respect to prevention, diagnosis, treatment, patient monitoring, prognosis, and/or drug design.

For example, dangerous infectious diseases for which no optimal medical management methods are available include acquired immunodeficiency syndrome (AIDS) caused by human immunodeficiency virus (HIV), influenza, malaria, hepatitis, tuberculosis, cholera, Ebola virus infection, and severe acute respiratory syndrome (SARS). Such diseases are collectively responsible for millions of deaths worldwide each year. Ominously, diseases such as AIDS, and antibiotic-resistant bacterial and mycobacterial infections, such as antibiotic-resistant staphylococcal and tuberculosis infections, respectively, to which there are no satisfactory cures for most affected individuals, are on the increase. Also of concern is the widely perceived and anticipated threat of biological warfare using agents causing lethal infectious diseases, such as anthrax, smallpox, and bubonic plague. Hence, society is confronted with the challenge of vaccinating on relatively short notice large numbers of persons against such pathogens. Infectious diseases require better diagnostic discrimination between persons who will be susceptible to a particular vaccination and persons who will not respond. Certain infections can trigger autoimmune responses, and it is important to be able to diagnose persons who are destined to develop autoimmune diseases. With respect to vaccination strategies against infectious diseases, significant numbers of people have various degrees of immune malfunction—genetic, drug-induced, or acquired by infection or neoplasia—that could lead to serious complications upon exposure to live vaccines such as vaccinia. Idiosyncratic reactions to killed virus or viral subunit vaccines could also cause serious illness. Therefore, it has become essential to develop ways to survey the immune state of large numbers of people in a manner that is fast, reliable, safe and relatively inexpensive. In particular, it is necessary to be able to stratify individuals so as to predict the potential hazards of various vaccinations.

Autoimmune diseases represent a large group of highly debilitating and/or lethal diseases which includes such widespread and devastating diseases as rheumatoid arthritis, type I diabetes and multiple sclerosis. Traditionally, immunologic diagnosis and prognosis has been based on an attempt to correlate each condition with a specific immune reactivity, such as an antibody or a T-lymphocyte response to a single antigen specific for the disease entity. This approach has been largely unsuccessful for various reasons, such as the absence of specific antigens serving as markers of the disease. In the case of autoimmune diseases, this approach has been unsuccessful due to, for example, immunity to multiple self-antigens, as exemplified by type I diabetes which may be associated with a dozen different antigens, and due to the fact that a significant number of healthy persons may manifest antibodies or T-lymphocyte reactivities to self-antigens targeted in autoimmune diseases, such as insulin, DNA, myelin basic protein, thyroglobulin and others. For this reason, false positive tests are not uncommon. Hence, there is a real danger of making a false diagnosis based on the determination of a given immune reactivity. Novel approaches, therefore, are needed to support the diagnoses of specific immune conditions in a way that would justify specific therapeutic decisions.

Malignant diseases such as breast cancer, lung cancer, colorectal cancer, melanoma and prostate cancer are a tremendous medical and economic burden, particularly in industrialized populations. The immunotherapy of cancer is a situation in which it would be advantageous to classify persons with different types of immune reactivities to self-antigens; many, if not most, tumor-associated antigens are self-antigens. Thus, it is important in the design of therapeutic tumor vaccines to know what kind of immune potential is present in the patient. In individuals who have received chemotherapy and stem cell transplants for leukemias and other cancers, the monitoring of the overall breadth of the recovering immune system becomes crucially important. An immune system with a broader repertoire reflects one with more potential to combat infections.

Transplantation related diseases such as graft rejection and graft-versus-host disease are major causes of failure of therapeutic transplantation, a medical procedure of last resort broadly practiced for treating numerous life-threatening diseases, such as cardiac, renal, pulmonary, hepatic and pancreatic failure.

Allergic diseases, such as allergy to seasonal pollens, ragweed, dust mites, pet fur, cosmetics, and various foods are significantly debilitating to a large proportion of the population, can be fatal, and are of great economic significance due to the large market for allergy drugs.

The need for optimal methods of monitoring immune responses and disease progression is acutely felt in the pharmaceutical industry in the development of new therapeutic biological agents and drugs. Autoimmune and degenerative diseases are intrinsically difficult to deal with pharmaceutically. Not only are these diseases chronic, but the individual patients enrolled in treatment trials tending to be in different states of responsiveness. Thus it is difficult to devise a single dose of a drug and a treatment schedule that will be optimal for each individual. Some individuals need larger or smaller doses, or more or less frequent administration for an optimal response. Thus it is all too easy to miss the mark, and even effective drugs have failed to reach statistical significance in trials. Indeed, it is costly and hazardous to risk the success of a new drug on a long-term trial of one or a few doses or modes of administration. The industry critically requires predictive markers to stratify individuals and design trials based providing critical immunologic information regarding the response of the test individuals. Clinical trials of anti-inflammatory drugs have focused on the disease as the only endpoint, and have failed to monitor the cause of the disease. Hence, methods of characterizing antigenic specificities of the immune system could provide the information needed to optimize effectiveness and save time in arriving at dosing and other variables.

Hence, there is an urgent need for novel and improved methods for facilitating optimal performance of various aspects of medical management of a vast range of antigen associated diseases.

The adaptive immune response of vertebrates allows specific response to a huge variety of antigens and pathogens. This response is based on the existence of B and T lymphocytes, that exert their function through B cell receptors (BCR) and T cell receptors (TCR) which are assembled through somatic gene recombination during B and T cell development. BCR and TCR are bound to the membrane of B and T lymphocytes together with coreceptors, that mediate specific signals after recognition of ligands. In addition B lymphocytes can secrete BCR in the form of specific antibodies, that are also mediators of immune reactions. Due to the recombination (also known as the V(D)J recombination) a high variability of BCR and TCR is possible, that is orders of magnitude higher than the number of lymphocytes in vertebrates.

B lymphocytes are the primary mediators of humoral immunity by productions of antibodies which are able to specifically bund to foreign invaders like viruses, parasites and bacteria and initiate their destruction. Antibodies are globular proteins that circulate in blood, lymphatic and bodily fluid. The humoral immune response is based on the recognition of antigen by the antibody. In the destruction of antigen, the antibody fulfills three main functions: (i) the activation of the major effector of the humoral branch, the complement system, a system based on various proteins; (ii) the binding to antigens, thus eliminating their capacity to harm; and (iii) the recognition by Fc receptors on professional phagocytic cells. The antigen is first recognized by membrane attached antibodies on the surface of a specific B cell. It is then endocytosed and presented on a class II MHC activating T-helper 2 cell. Other antigen presenting cell like dendritic cells or macrophages can also activate T-helper 2 cells. Afterwards, they will attach and stimulate B-cells by releasing cytokines such as IL-4 to initiate development into plasma cells. These plasma cells produce and secrete significant quantities of secreted antibodies, which bind to antigen either free in solution or on the surface of a foreign cell, forming a precipitate and causing a conformational change in the antibody Fc segment. This change allows the complement system to initiate lysis of the foreign cell in a cascade that begins with the binding of the antibody to a microbial surface antigen. Otherwise, if the antigen is not membrane-attached but precipitated by antibodies, “a bystander lysis” occurs, killing a vital cell. In addition cleavage products of proteins of the complement system serve as opsonins, which are responsible for recruiting neutrophils and macrophages. This initiates further sensitization of the immune system against the foreign antigen and an inflammatory response. Along with the function of antibodies as complement system activator, they also serve as opsonins themselves, recruiting neutrophils.

As the efficiency of a mammal to mount a humoral immune response is dependent on specific antibodies, the complete collection of expressed immunoglobulins (i.e., immunoglobulin repertoire) is a determinant of the immune status of the organism. However, as V(D)J recombination happens through somatic rearrangements of distinct genomic loci in B and T cells, also the collection of genomic V(D)J rearrangements of a vertebrate can be called a “repertoire”.

T-lymphocytes are the primary mediators of cellular immunity in humans, occupying an essential role in immune responses to infectious agents (e.g., viruses and bacteria) and in the body's natural defenses against neoplastic diseases. Likewise, T lymphocytes play a central role in acute graft versus host disease, wherein the immune system of host attacks implanted tissue from a foreign host, in autoimmune disorders, in hypersensitivity, in degenerative nervous system diseases and many other conditions. A T cell immune response is characterized by a T cell (or more) recognizing a particular antigen, secreting growth promoting cytokines and undergoing monoclonal (or oligoclonal) expansion to provide additional T cells to recognize and eliminate the foreign antigen.

Each T cell and its progeny are unique by virtue of a structurally unique T cell receptor (TCR), which recognizes a complementary structurally unique antigen. In general, T cells produce either of two types of TCR consisting of αβ and γδ. TCR αβ is found on 95% of lymphocytes. It is synthesized later in T-cell development than γδ. The TCR αβ is responsible for helper T cell function in cell mediated immunity and for killer T cell function in cell-mediated immunity. TCRs recognize a peptide in a groove on the surface of a MHC protein. The result of this specific interaction is signaling through the CD3 complex. Depending upon the stage of differentiation of the T cell and on the co-stimulatory signal, this can lead to T cell proliferation, to T cell effector function, to T cell anergy or to T cell death.

Diversity in TCR is generated through somatic recombination at the TCR loci. This recombination involves three different segment types V(D)J segments, resembling recombination in immunoglobulins. Additional diversity is generated at the junction of the segments during the recombination process. The organization of TCR α resembles that of Ig κ, with V genes separated from a cluster of J segments that precedes a single C chain. In addition to the α segmens, this locus also contains δ segments. The organization of TCR β is similar to the heavy chain of immunoglobulins, with V genes separated from two clusters each containing a D segment, several J segments and a C gene. Within the TCR α and β chain variable regions are hypervariable regions similar to those found in immunoglobulins, where they form the principal points of contact with antigen and thus are referred to as CDR (complementarity determining regions). Based on analogy with immunoglobulins, these TCR hypervariable regions are thought to loop out connecting β-sheet TCR framework sequences. Two CDRs (CDR1 and CDR2) are postulated to contact predominantly MHC peptide sequences, whereas a third, centrally-located CDR (CDR3) is believed to contact peptide bound in the MHC antigen binding groove.

The characterization of T cell responses in normal physiological and pathological situations, including auto-immunity, response to infectious agents, alloimmunity and tumor immunity, is a key to understand disease control by the immune system and is beginning to play an important role in many clinical situations.

The totality of BCRs and TCRs being expressed by a vertebrate at a certain point in time, i.e., the vertebrate immune repertoire, mirrors the vertebrate's immune status. Hence, from a concise analysis of a vertebrate immune gene repertoire, one can draw conclusions on the immune status and on the susceptibility to diseases. In addition, ongoing diseases and inflammatory reactions can be assessed via the immune gene repertoire, and decisions for treatment can be concluded.

Various immunoglobulin repertoire analyses have been performed in the past, and have shown that a change in the Ig repertoire can be related to different physiological stages of the organism. More specifically it was found that diseases like sarcoidosis, hepatitis, multiple sclerosis, lymphomas and graft versus host disease are associated with a shift in the Ig repertoire.

Thus for example, a restricted variable region usage has been shown in different physiological states associated with different developmental stages [Davidkova et al., Scand J Immunol, 45:62 (1997)], malignancies [Kipps T J et al., Proc Natl Acad Sci USA, 86:5913 (1989); Kotlan (2003) Hum. Antibodies 12:113-21; Messmer (2004) 200:519-25; Tobin (2004) Leuk. Lymph. 45:221-8; Ritgen (2003) Blood 101:2049-53; Kienle (2003) Blood 102:3003-9], autoimmunity [Risitano (2004) Lancet 364:308-9; Fraser (2003) Arthritis Res. Ther. 5:R114-21; Demoulins (2003) Neurobiol. Dis. 14:470-82; Mockridge (2004) Autoimmunity 37(1):9-15; Mockridge (2004) 37:9-15; Sade (2003) Allergy 58:430-4; Pascual (1990) Int. Rev. Immunol. 5:231] and in infectious diseases [Ohlin and Zouali (2003) Mol. Immunol. 40: 1-11; Gasparotto (2002) Leukemia and Lymphoma 43:747].

However, all previous repertoire analyses were hampered by their experimental design, which did not allow for high throughput analysis. Previous analyses were performed using colony hybridizationto filters, sequencing or complementarity determining region (CDR) spectratyping. Those techniques were very laborious and did not allow to assess and compare the Ig and TCR repertoire of a statistically significant number of individuals.

DNA microarrays, also known as “DNA Chips”, are a potentially powerful technology for improving diagnostic classification, treatment selection and development of therapeutics.

In the past several years, this technology has emerged, promising to monitor the whole genome on a single chip, allowing better identification of drug protein candidates and gene expression profiling in different disease state conditions, especially in the diagnosis of a vast platform of malignancies for example in the diagnosis of haematological malignancies. It enables the analysis of RNA expression by clonal populations of leukemia and lymphoma cells on a genome-wide scale (Best Pract Res Clin Haematol. December; 16(4):645-52, 2003). Furthermore, by monitoring multiple genes in parallel DNA chip technology allows the identification of genes that are differentially expressed in malignant and normal tissues and classify theses genes as “signatures”, of the disease state. Often, these signatures are impossible to obtain from tracking changes in the expression of individual genes, which can be subtle or variable.

Microarray for gene expression analysis is based on labeled cDNA or cRNA targets derived from the mRNA of an experimental sample are hybridized to nucleic acid probes attached to the solid support. By monitoring the amount of label associated with each DNA location, it is possible to infer the abundance of each mRNA species represented (de Saizieu, A., et al., Nature Biotech. 16: 45-48, 1998; and U.S. Pat. No. 6,410,229). However, this technology requires prior knowledge of the gene sequence or customization for each new application. In the case of antibodies or TCRs due to the high variability of the sequence, this approach is not practical since every specimen is of potential interest and the number of potential types of antibody and TCR sequences is beyond the capacity of the gene expression profiling DNA chip technology.

Although sequence information has already provided accelerated knowledge and potential resolution of diverse biological, medical and therapeutic research problems, in practice, actual sequencing requires large number of base pairs in order to obtain a reliable sequence.

Further challenges arise if sequencing projects are extended to include the determination of the genomic sequences of characteristic individuals or species of organisms, especially those that have economic, social or medical importance. Such sequencing projects would advance not only our understanding of the evolution of organisms and the evolution of biochemical processes, but would also further the detection, treatment and understanding of disease, and would aid agriculture, the food industry and biotechnology in general. However beneficial the results of such projects would be, their successful completion requires the development of a new, rapid, reproducible and reliable sequencing method such as those described in this invention.

Accurate DNA sequencing is a crucial procedure in modern biological, medical and agricultural research. Traditionally DNA sequencing has been done by direct, base-by-base analysis, with each new base determination built on the results of many previous sequencing steps.

Direct sequencing techniques involve a variety of synthesis, degradation, or separation techniques, and include the traditional Sanger, pyrosequencing and exonuclease methods, as well as direct visualization approaches (see for example US patent application 2002/0009727; Shi, Clinical Chemistry 47:164-172, 2001; and Drmanac R., et al., Adv Biochem Engineering/Biotech 77:75-101, 2002).

In the Sanger method, DNA synthesis is randomly terminated at each base pair, creating a wide range of fragments that are then separated by gel according to length and scored. In the pyrosequencing method, polymerase-guided incorporation of each base is detected by measuring the pyrophosphate released in consecutive cycles. In the exonuclease approach, a single molecule of target DNA synthesized with fluorescent-tagged bases is degraded by exonuclease. The consecutively released nucleotides are then scored with a very sensitive fluorescence detector.

Indirect sequencing method such as “sequencing by hybridization” (SBH) is designed to sequence a target nucleic acid by allowing the identification of complementary sequences in the target nucleic acid by utilizing n-mers probes (all possible probes of length n). This method comprises the “universal microarray” approach, in which probes are designed using simple combinatorial and statistical principles, without guidance from prior knowledge of any specific gene or DNA sequence, was developed by several companies (Hyseq Inc., Genometrix Inc.) (Advances in Biochemical Engineering Biotechnology 77:76-101; and U.S. Pat. No. 5,695,940). The method relies on the process, in which oligonucleotide probes hybridize preferentially with entirely complementary and homologous nucleic acid targets are described. Using these hybridization conditions, overlapping oligonucleotide probes associate with a target nucleic acid. Following washes, positive hybridization signals are used to assemble the sequence of a given nucleic acid fragment. Representative target nucleic acids are applied as dots. Up to 4⁸ or ˜66,000 probes of the type (A,T,C,G)(A,T,C,G)N8(A,T,C,G) are used to determine sequence information by simultaneous hybridization with nucleic acid molecules bound to a filter. Additional hybridization conditions are provided that allow stringent hybridization of 6-10 nucleotide long oligomers which extends the utility of the invention. A computer process determines the information sequence of the target nucleic acid which can include targets with the complexity of mammalian genomes. Sequence generation can be obtained for a large complex mammalian genome in a single process (U.S. Pat. No. 5,525,464).

U.S. Pat. No. 5,202,231, U.S. Pat. No. 5,525,464 and WO 95/09248 disclose additional aspects of sequencing by hybridization. However, due to the repetitive nature of genomic DNA, this approach has not been proven as competitive as current biochemical sequencing methods. It has been used successfully for finding mutations (Gerry, et al J Mol Biol 292: 251-62, 1999).

Various twists of the SBH technology such as the use of universal bases are claiming improvement of the SBH process (Frieze, et al. J Comput Biol. 6: 361-368, 1999; Preparata F P et al., Comput Biol. 7: 621-30, 2000; and US Patent application 2003/0036073); Non-universal probes in SBH is used to detect the presence of known sequences in a sample, determine presence/absence and position of mutations or discover novel SNPs or single base mutations; another development of SBH is the combinatorial ligation of two sets of short probes to create long probes which allow the detection of long complementary sequences in target DNAs without the use of large sets of long probes (termed combinatorial SBH). For example, two sets of 4096 6 mers can be combined to score over 16×10⁶ 12 mers. Only a small number of manufactured probes are needed initially and each probe can be synthesized individually by standard techniques to create large quantities of very high quality probes for use in millions of assays. By requiring simultaneous initial hybridization of two short probes instead of a single long one, the overall hybridization specificity is increased substantially. In combinatorial SBH, one of the two sets of probes is bound to the support another labeled set is in the hybridizing solution. Both sets are hybridized to the target DNA in the presence of DNA ligase. Bound and labeled probes that hybridize to the target at precisely adjacent positions are ligated to form a labeled, support-bound long probe. Only these labeled, support bound long probes formed by ligation are then scored by an appropriate detection device. Using combinatorial SBH researchers were able to perform comparative sequencing, re-sequencing and SNP discovery experiments on several human, bacterial and viral genes (see Drmanac (2002) Advances in Biochemical Engineering/Biotechnology 77: 75-101). However, to date combinatorial SBH technology has not been used to high throughput screen large sequence deviations such as those featuring antigen receptors.

There is thus an unmet need for improved high throughput methods for sequencing and profiling expressed genes belonging to the immunoglobulin gene superfamily, particularly immunoglobulins and T cell receptors.

### SUMMARY OF THE INVENTION

The present invention advantageously provides methods of sequencing antibodies and T cell receptors, generating profiles thereof, and correlating the data generated with states of disease or health.

According to one aspect of the present invention there is provided a method of sequencing a population of polynucleotides encoding antibodies or T-cell receptors, the method comprising: (a) contacting a plurality of oligonucleotides of known sequences, at least a portion of the plurality of oligonucleotides having a partial sequence similarity, with the population of polynucleotides under conditions allowing a formation of hybridization duplexes between at least a portion of the plurality of oligonucleotides and the population of polynucleotides; (b) quantitavely detecting oligonucleotides involved in the formation of the hybridization duplexes; and (c) compiling a set of sequences of the population of polynucleotides by: (i) identifying oligonucleotides involved in the formation of the hybridization duplexes in a quantum above a predetermined threshold, so as to define a population of positively hybridizing oligonucleotides; (ii) identifying germline sequences of germline segments in at least a portion of the positively hybridizing oligonucleotides or in at least a portion of the oligonucleotides involved in the formation of the hybridization complexes; (iii) identifying positively hybridizing oligonucleotides overlapping with the germline sequences, thereby identifying germline junction sequences; and (iv) assembling sequence information obtained from steps (ii) and (iii), thereby compiling the set of sequences of the population of polynucleotides; thereby sequencing the population of polynucleotides encoding the antibodies or T-cell receptors.

According to another aspect of the present invention there is provided a method of quantifying an expression of a population of polynucleotides encoding antibodies or T-cell receptors, the method comprising: (a) contacting a plurality of oligonucleotides of known sequences, at least a portion of the plurality of oligonucleotides having a partial sequence similarity, with the population of polynucleotides under conditions allowing a formation of hybridization duplexes between at least a portion of the plurality of oligonucleotides and the population of polynucleotides; (b) quantitavely detecting oligonucleotides involved in the formation of the hybridization duplexes; and (c) compiling a set of sequences of the population of polynucleotides by: (i) identifying oligonucleotides involved in the formation of the hybridization duplexes in a quantom above a predetermined threshold, so as to define a population of positively hybridizing oligonucleotides; (ii) identifying germline sequences of germline segments in at least a portion of the positively hybridizing oligonucleotides or in at least a portion of the oligonucleotides involved in the formation of the hybridization complexes; (iii) identifying positively hybridizing oligonucleotides overlapping with the germline sequences, thereby identifying germline junction sequences; (iv) assembling sequence information obtained from steps (ii) and (iii), thereby compiling the set of sequences of the population of polynucleotides, and (d) determining a level of each set of the compiled set of sequences of step (iv) in the population of polynucleotides, thereby quantifying the expression of the population of polynucleotides encoding the antibodies or T-cell receptors.

According to further features in preferred embodiments of the invention described below, step (ii) further comprises identifying oligonucleotides of non-redundant germline sequences in the germline segment.

According to still further features in the described preferred embodiments each oligonucleotide of the at least a portion of the plurality of oligonucleotides is selected to hybridize with a known germline segment and an unknown sequence.

According to still further features in the described preferred embodiments the plurality of oligonucleotides are collectively selected to hybridize with all germline segments of the population of polynucleotides.

According to still further features in the described preferred embodiments the plurality of oligonucleotides is selected to non-redundantly hybridize with the germline segments of the population of polynucleotides.

According to still further features in the described preferred embodiments the plurality of oligonucleotides comprise soluble oligonucleotides and/or oligonucleotides attached to a solid support in an addressable location.

According to still further features in the described preferred embodiments the soluble oligonucleotides comprise a label.

According to still further features in the described preferred embodiments the population of polynucleotides comprise a label.

According to still further features in the described preferred embodiments each oligonucleotide of the plurality of oligonucleotides is 5-40 nucleotides in length.

According to still further features in the described preferred embodiments the population of polynucleotides comprise RNA molecules.

According to still further features in the described preferred embodiments the population of polynucleotides comprise DNA molecules.

According to still further features in the described preferred embodiments the method further comprising, prior to step (a), amplifying selected segments of the polynucleotides encoding the antibodies or the T-cell receptors, the selected segments encoding variable regions of the antibodies or the T cell receptors.

According to still further features in the described preferred embodiments the variable regions comprise variable regions of heavy chains of antibodies.

According to still further features in the described preferred embodiments the variable regions comprise variable regions of light chains of antibodies.

According to still further features in the described preferred embodiments the variable regions comprise variable regions of TCRα.

According to still further features in the described preferred embodiments the variable regions comprise variable regions of TCRβ.

According to still further features in the described preferred embodiments the variable regions comprise variable regions of TCR γ.

According to still further features in the described preferred embodiments the variable regions comprise variable regions of TCR δ.

According to still further features in the described preferred embodiments the method further comprises storing the set of sequences of the population of polynucleotides on a computer readable storage medium.

According to yet another aspect of the present invention there is provided an oligonucleotide library for sequencing by hybridization of polynucleotides encoding variable regions of antibodies or T cell receptors, the library consisting essentially of: (i) a set of overlapping oligonucleotides collectively selected to hybridize with all germline segments encoding the variable regions of the antibodies or T cell receptors under conditions allowing formation of hybridization duplexes between the set of overlapping oligonucleotides and the polynucleotides; and (ii) a variant set of oligonucleotides of the overlapping oligonucleotides which comprises (G,C,T,A) base variation in at least one position of the overlapping oligonucleotides; wherein oligonucleotides of the sets of overlapping oligonucleotides and the variant set of oligonucleotides are N bases in length and the overlapping is of at least N−(N−1) and whereas N is an integer equal or greater than 5.

According to still further features in the described preferred embodiments the base variation representing percent variation in corresponding complementary subsequence of the variable regions of the antibodies or T cell receptors.

According to still further features in the described preferred embodiments the subsequence of the variable regions of the antibodies or T cell receptor is a framework subsequence and whereas the percent variation is below 17%.

According to still further features in the described preferred embodiments the subsequence of the variable regions of the antibodies or T cell receptor is a CDR subsequence and whereas the percent variation is below 30%.

According to still further features in the described preferred embodiments the overlapping oligonucleotides are selected capable of hybridizing with non-redundant germline sequences of the germline segments.

According to still further features in the described preferred embodiments N is 5-40.

The present invention successfully addresses the shortcomings of the presently known configurations by providing oligonucleotide libraries, arrays thereof and methods of using same for quantifying and profiling of antibodies and T-cell receptors gene expression.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1—A flowchart of the invention process.

FIG. 2—Compilation with overlapping positively labeled probes.

FIG. 3—Examples of multiple sample experiment.

FIG. 4—Scheme illustrating end labeled plus and minus strand.

FIG. 5—Scheme illustrating PCR amplification of specific regions within the target sequence.

FIG. 6—A bar graph correlating probe length with redundancy thereof in human germline segments.

FIG. 7 shows an example of oligonucleotide sequences selected to span a specific VD junction.

FIG. 8 shows the nucleic acid sequence of Homo sapiens anti-HIV-1 gp120 immunoglobulin heavy chain variable region mRNA, partial cds. Frameworks regions are underlined (FR1-FR4); CDR regions are shown in bold (CDR1-3).

FIG. 9 is a multiple alignment of AY056842.1 with: VH3-64, D3-22 and JH3.

### DETAILED DESCRIPTION OF THE PREFERRED EMBODIMENTS

The present invention is of oligonucleotides and arrays thereof which can be used to sequence polynucleotide populations of antibodies and T-cell receptors. Specifically, sequence information obtained by the present invention can be used to elucidate a T-cell receptor and/or antibody repertoire of an individual and elucidate the immune status of the individual (e.g., previous or current diseases, protection against future diseases, prediction of disease progression). Information generated according to the teachings of the present invention can be used to identify protective antibodies or T-cell receptors and to identify pathogenic antigens.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

It is well recognized that elucidating the entire repertoire of antibodies and T-Cell receptors of a subject portraits a current immune status thereof. Such knowledge is useful for diagnosis, disease management and prediction of antigen associated diseases as well as for the identification of protective antibodies and disease causing antigens, which may be used for designing new therapeutic drugs and proteins for antigen-associated diseases.

Various immunoglobulin repertoire analyses have been performed in the past and have shown that a change in immunoglobulin repertoire can be related to different physiological stages of an organism.

However, all previous repertoire analyses were hampered by their experimental design which did not allow for high throughput analysis. Previous analyses included colony hybridization to filters, sequencing or CDR spectratyping. Those techniques were very laborious and did not allow assessing and comparing the Ig and TCR repertoire of a significant number of individuals.

DNA microarrays (also referred to herein as DNA chips and arrays), have emerged as a robust technology for monitoring a whole genome on a single chip, allowing better identification of drug protein candidates and gene expression profiling in different disease state conditions, especially in the diagnosis of a vast platform of malignancies for example in the diagnosis of haematological malignancies.

In the case of antibodies or TCRs due to the high variability of their sequence, this approach is not practical since every specimen is of potential interest and the number of potential types of antibody and TCR sequences is beyond the capacity of the gene expression profiling DNA chip technology.

While reducing the present invention to practice, the present inventor uncovered that by making use of known sequence data pertaining to conserved sequences of antibodies and TCRs, arrangement of germline segments, as well as recombination and mutations thereof it is possible to amplify and sequence the variable regions of an entire antibody or TCR repertoire of an organism.

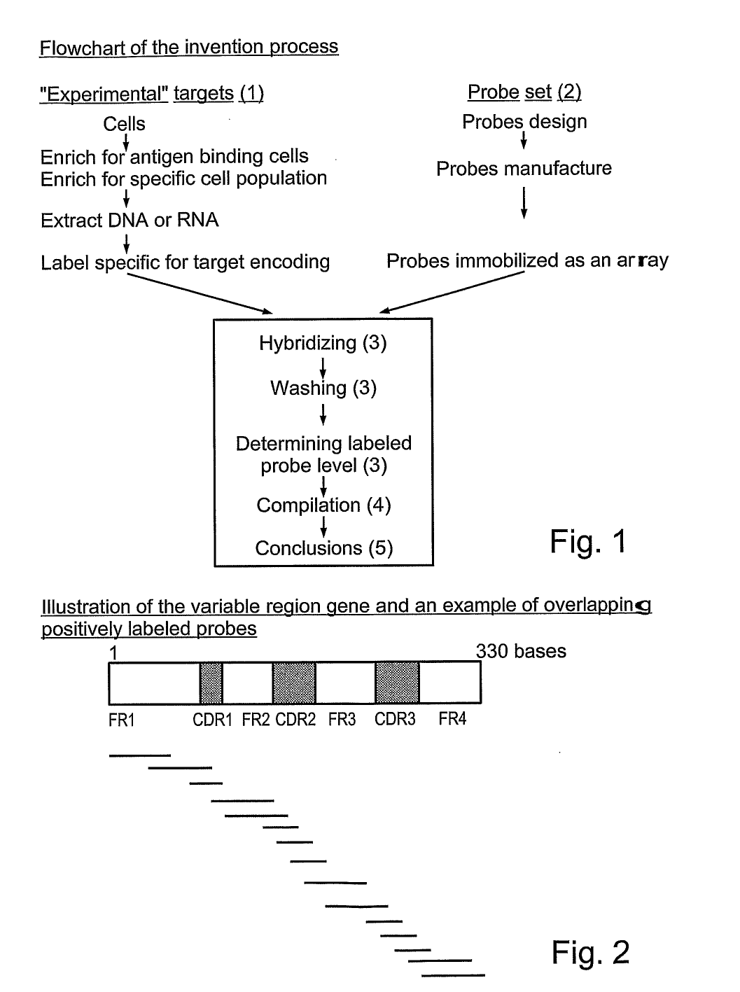

Thus, according to one aspect of the present invention there is provided a method of sequencing a population of polynucleotides encoding antibodies or T-cell receptors (see FIG. 1).

As used herein the term “sequencing” refers to determining the order of nucleotides (base sequences) in a DNA or RNA molecule encoding an antigen binding domain (i.e., a variable region) of a TCR or an antibody [V(D)J].

As used herein the term “antibodies” refers to membrane anchored B cell receptors (BCRs) or soluble secreted forms thereof (antibodies).

As used herein “a population of polynucleotides encoding antibodies or T cell receptors (TCRs)” refers to DNA and/or RNA molecules which encode for antibodies or TCRs. Polynucleotides of the present invention (also referred to herein as “target nucleic acids”), may be in a single- or double stranded form.

It will be appreciated that there is a certain advantage in using RNA as the target nucleic acid for the analyses of the present invention. If genomic DNA is used, one needs to differ between the two alleles for the target genes, since only one is expressed and rearranged. In addition the invention is aimed to cells expressing the target nucleic acids and not to non-mature forms of the targets. Therefore, when using DNA as the biological material, previous enrichment for cells expressing the target genes or CD markers should be considered. This enrichment will select the cells that contain rearranged target gene.

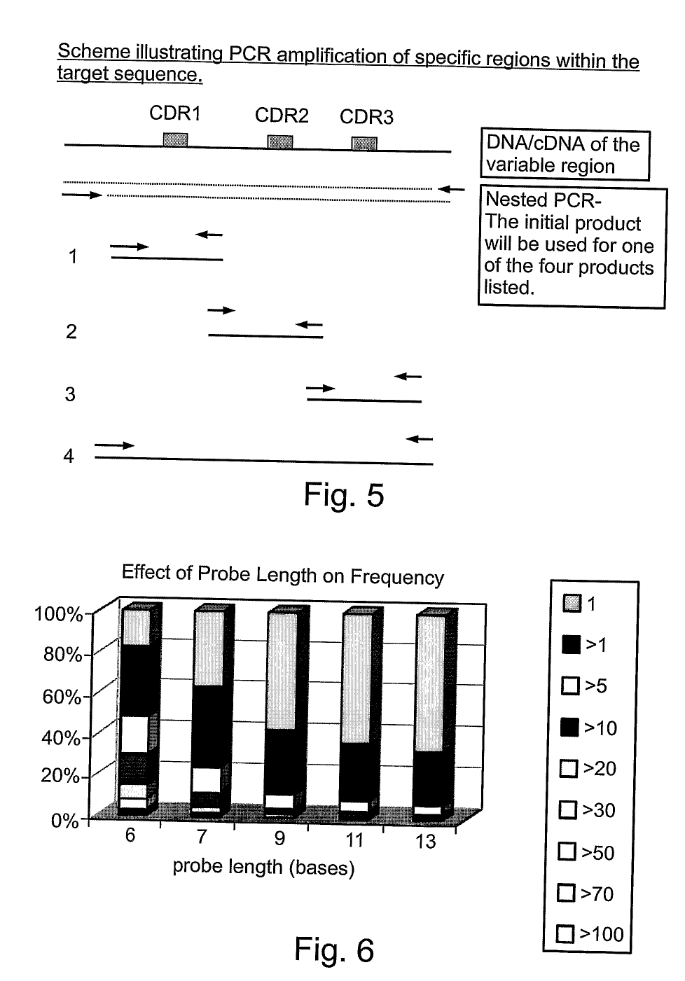

Preferably, the target mucleic acids encode for the variable regions (CDRs+frameworks, see FIG. 2) regions of antibodies (heavy and light) and TCRs (αβδγ). Focusing on such sequences may be effected by PCR or RT-PCR reactions using specific primers, such as for example, primers which are directed to the conserved leader sequence and a degenerate primer [Sims et al. (2001) Immunol. 15:167:1935-44; see also Examples section hereinbelow and FIG. 5].

Preferably the plus and minus strands of the population of nucleic acids are analyzed by the present invention. Depending on the experimental design, the hybridization can be to the same array or to two duplicated arrays (further described hereinbelow). If the hybridization is to the same array, two different labels are used such as cy3 and cy5—one for labeling the plus strand and the other for the minus strand (FIG. 4). This approach has several advantages:

a) Each sequence will be confirmed since both plus and minus strands are used.

b) There is a reduction of the impact of false negative and of the impact of false positive. False negative is the case that oligos that should have produced a significant hybridization signal and did not. False positive is the case that oligos that produced a significant hybridization signal yet are not complementary to target sequence. The likelihood of false positive and false negative is, overall, reduced since they need to insert the same effect on the plus and minus strands, in order to cause a real problem.

c) This experimental setting, allows oligos to function as internal controls to themselves. Thus, probes that bind weakly to the target nucleic acids will also have a weak non-specific binding with the opposite strand.

The population of polynucleotides encoding antibodies or T cell receptors (nucleic acid targets) may be a homogeneous population (e.g., obtained from a hybridoma, or any cell transformed to express an antibody or a TCR) or a heterogeneous population. The population of antibodies or TCRs of the present invention may be obtained from biological samples including same. As used herein the phrase “biological sample” refers to tissues and cells obtained from bodily fluids and tissues such as, serum, sputum, blood, blood cells (e.g., white cells), tissue or fine needle biopsy samples, urine, peritoneal fluid, and pleural fluid, or cells therefrom. Biological samples may also include sections of tissues such as frozen sections such as those taken for histological purposes. Biological samples also refer to cells which were genetically manipulated to express an antibody or a TCR. Thus, for example, polynucleotides encoding for TCRs may be obtained from T-lymphocytes, polynucleotides encoding for BCRs may be obtained from B-lymphocytes and plasma cells.

The method according to this aspect of the present invention comprising (a) contacting a plurality of oligonucleotides (also termed as probes) of known sequences, at least a portion of the plurality of oligonucleotides having a partial sequence similarity, with the population of polynucleotides under conditions allowing a formation of hybridization duplexes between at least a portion of the plurality of oligonucleotides and the population of polynucleotides; (b) quantitavely detecting oligonucleotides involved in the formation of the hybridization duplexes; and (c) compiling a set of sequences of the population of polynucleotides as further described hereinbelow, thereby sequencing the population of polynucleotides encoding the antibodies or T-cell receptors.

As used herein the term “oligonucleotide” or “probe” refers to a single-stranded polymer of ribonucleic acid (RNA) or deoxyribonucleic acid (DNA) or mimetics thereof. This term includes oligonucleotides composed of naturally occurring bases, sugars, and covalent internucleoside linkages (e.g., backbone), as well as oligonucleotides having non-naturally occurring portions, which function similarly to respective naturally occurring portions. The oligonucleotide population of the present invention is capable of binding to a complementary sequence of the target polynucleotide through one or more types of chemical bonds, of complementary base pairing, usually through hydrogen bond formation.

Oligonucleotides designed according to the teachings of the present invention can be generated according to any oligonucleotide synthesis method known in the art, such as enzymatic synthesis or solid-phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example: Sambrook, J. and Russell, D. W. (2001), “Molecular Cloning: A Laboratory Manual”; Ausubel, R. M. et al., eds. (1994, 1989), “Current Protocols in Molecular Biology,” Volumes I-III, John Wiley & Sons, Baltimore, Md.; Perbal, B. (1988), “A Practical Guide to Molecular Cloning,” John Wiley & Sons, New York; and Gait, M. J., ed. (1984), “Oligonucleotide Synthesis”; utilizing solid-phase chemistry, e.g. cyanoethyl phosphoramidite followed by deprotection, desalting, and purification by, for example, an automated trityl-on method or HPLC.

The oligonucleotides of the present invention are of at least about 5, at least about 6, at least about 7, at least about 8, at least about 9, at least about 10, at least about 11, at least about 12, at least about 15, at least about 17, at least about 18, at least about 19, at least about 20, at least about 22, at least about 25, at least about 30 or at least about 40 bases at least a portion of which having at least partial sequence similarity with the polynucleotides of the present invention.

To detect long complementary sequences in the target nucleic acid without the use of large sets of long probes, two sets or more of short probes are preferably used (as in combinatorial SBH described in the Background section). Such probes can be of varying lengths such as a 6+5 combination.

The oligonucleotides of the present invention may comprise heterocylic nucleosides consisting of purines and the pyrimidines bases, bonded in a 3′-to-5′ phosphodiester linkage.

Oligonucleotides of the present invention may be modified either in backbone, internucleoside linkages, or bases.

Specific examples of preferred oligonucleotides useful according to this aspect of the present invention include oligonucleotides containing modified backbones or non-natural internucleoside linkages. Oligonucleotides having modified backbones include those that retain a phosphorus atom in the backbone, as disclosed in U.S. Pat. Nos. 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

The plurality of oligonucleotide of the present invention is preferably attached to a solid support such as a microarray (also referred to herein as an array or a chip). When combinatorial hybridization is used one set of probes is attached to the solid support while another set is soluble and added to the hybridization solution.

Ample guidance for obtaining and utilizing a probe array for suitably practicing the method of the present invention is provided in the literature of the art (for example, refer to: U.S. Pat. No. 6,551,784; U.S. Pat. No. 6,251,601; Forster et al., 2003. J. Endocrinol. 178:195-204; Howbrook et al., 2003. Drug Discov Today 8:642-51; Xiang et al., 2003. Curr Opin Drug Discov Devel. 6:384; Hardiman G., 2003. Pharmacogenomics 4:251). Custom designed arrays can be purchased from commercial suppliers [for example, Callida Genomics, Affymetrix, Santa Clara, USA; or Agilent Technologies, Palo Alto, USA).

The probe array may include the plurality of addressable locations at any of various surface densities, depending on the application and purpose.

Oligonucleotide probes of the present invention are selected able to cover the junctions between the germline segments composing the variable region of antibodies or TCRs. Table 2 demonstrates the various combinatorial possibilities for the rearranged variable region. In the case of the light. Ig chain, the rearrangement is of the joining of VJ gene segments. In the case of the heavy chain the rearrangement is of the joining of VDJ gene segments (see Table 2). Variable number of bases can be deleted or added (0-15) at the junction.

There are several options in revealing the junctions sequence (one junction in the light chain and two junctions in the heavy chain):

1. Using a “true” universal chip that contains all possible combinations of n-mers (e.g., HyChip™, Callida Genomics, see Example 13 of the Examples section).

2. Selecting the more abundant representative sequences and for them designing specific PCR primers, the PCR product can be sequenced by conventional methods.

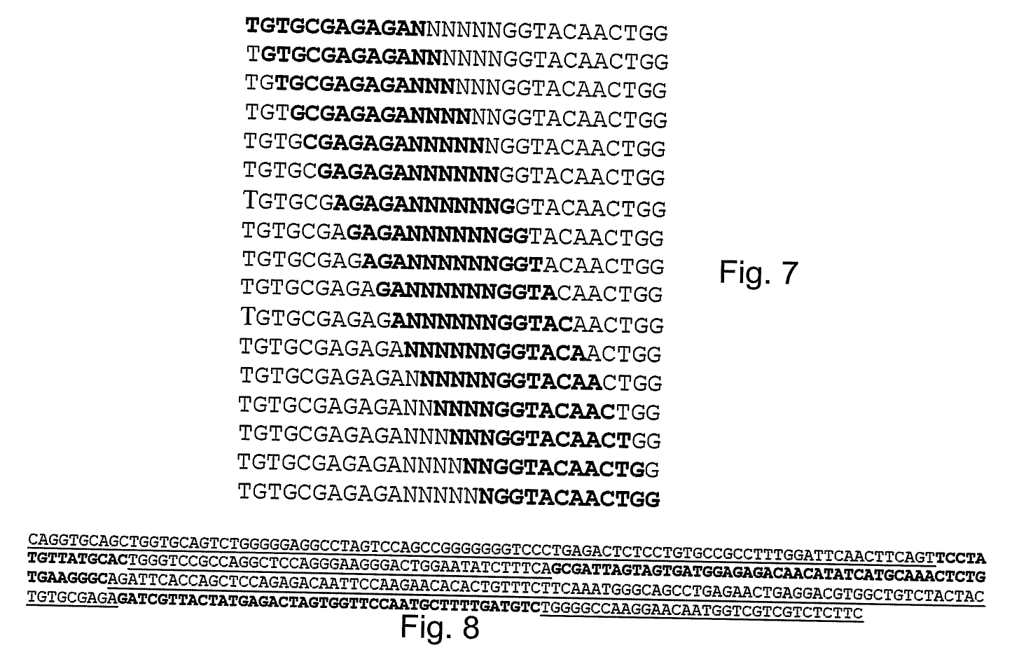

3. The junction can be revealed by oligos that posses partial overlap with the germline gene flanked by random sequence. This is illustrated in FIG. 7, where N symbolizes any one of the bases (ATGC).

When used in a cSBH configuration, soluble probes can be designed to hybridize to germline segments. For example, when 7-mer probes are used only 3367 different probes are needed (out of 16,000 theoretical combinations) to cover all the heavy germline genes.

Preferred guidelines for probe selection include: Selection of probes which are specific to a given germline segment. Measures are taken to ensure full coverage of the gene without opening of gaps. However probe redundancy may be reduced by allowing only minimal overlaps (e.g., a certain base need not be covered by more than one probe). This allows to reduce the number of probes and select probes which are more discriminative; In the junction area all possible overlapping probes are used to determine the junction sequence with high level of confidence.

According to a preferred embodiment of the present invention an oligonucleotide library for hybridization based sequencing of polynucleotides encoding variable regions of antibodies or T cell receptors is used. The library consisting essentially of: (i) a set of overlapping oligonucleotides collectively selected to hybridize with all germline segments encoding the variable regions of the antibodies or T cell receptors under conditions allowing formation of hybridization duplexes between the set of overlapping oligonucleotides and the polynucleotides; and (ii) a variant set of oligonucleotides of the overlapping oligonucleotides which comprises (G,C,T,A) base variation in at least one position of the overlapping oligonucleotides; wherein oligonucleotides of the sets of overlapping oligonucleotides and the variant set of oligonucleotides are N bases in length and the overlapping is of at least N−(N−1) and whereas N is an integer equal or greater than 5.

According to one preferred embodiment the base variation representing percent variation in corresponding complementary subsequence of the variable regions of the antibodies or T cell receptors.

According to another preferred embodiment the subsequence of the variable regions of the antibodies or T cell receptor is a framework subsequence and whereas the percent variation is below 17%.

According to yet another preferred embodiment the subsequence of the variable regions of the antibodies or T cell receptor is a CDR subsequence and whereas the percent variation is below 30%.

According to still another preferred embodiment N is 5-40.

The plurality of oligonucleotides of the present invention may also include control probes. Such control probe molecules may include normalization control probes, and/or expression level control probes and optionally negative control probes (which can define background signal).

Normalization control probes are probe molecules that are perfectly complementary to labeled reference oligonucleotides that are included in the hybridization solution. The signals obtained from the normalization control probes after hybridization provide a control for variations in hybridization conditions, label intensity, “reading” efficiency and other factors that may cause the signal of a perfect hybridization to vary between arrays. For example, signals, such as fluorescence intensity, read from all other probe molecules of the probe array are divided by the signal (e.g., fluorescence intensity) from the normalization control probes thereby normalizing the measurements.

Since hybridization efficiency varies with base composition and probe length, polynucleotide normalization control probes may be selected to reflect the average length of single stranded oligonucleotide probe molecules of the present invention, or multiple normalization control probes may be selected to cover a range of lengths of single stranded polynucleotide probe molecules of the present invention. Normalization control probes may be selected to reflect the base composition of the probe molecules of the probe set. Preferably, normalization control probes are incapable of substantially hybridizing with a target nucleotide sequence. Normalization control probes can be bound to various addressable locations the probe array to control for spatial variation in hybridization efficiently. Preferably, normalization control probes are located at the corners or edges of the array to control for edge effects, as well as in the middle of the array.

Different methods of normalization are known in the art. Examples include, but are not limited to total intensity normalization, LOWESS normalization, mean centering, ratio statistics, standard deviation regularization and more (Quackenbush J. Nat. Genet. 2002 December; 32 Suppl:496-501)

Expression level control probes are probe molecules that hybridize specifically with polynucleotides derived from housekeeping gene mRNA expressed in the cells from which nucleic acid target was retrieved, and may therefore be used to provide a normalization reference for comparing expression levels of different variants of the TCRs or antibodies. Suitable reference sequences include the sequences for constant regions, which can be used as expression level controls.

Background signal resulting from non-specific interaction (i.e., which is not a result of base complementation) between the target nucleic acids and the oligonucleotide probes and may be defined as the lowest hybridization signal detected in the system. Background signal may be adjusted to tolerable level (minimal signal/noise ratio) by modifying the hybridization buffer (e.g., detergent concentration), probe concentration and hybridization procedure (e.g., number of washes). For further details on determining background signal see U.S. Pat. No. 5,525,464. Negative control probes may be included and will preferably not bind the target nucleic acids. Such a control probe may be a poly run of a single nucleotide.

There are two parameters which influence the choice of probe length. The first is the success in obtaining hybridization results that show the required degree of discrimination. The second is the technological feasibility of synthesis of the required number of probes.

As mentioned hereinabove, the use of two sets of short probes in the framework of combinatorial SBH may be preferred since it allows the sequencing of long nucleic acid targets while employing relatively small number of probes [for further details see Drmanac R., et al., Adv Biochem Engineering/Biotech 77:75-101, (2002)]. In this case at least one of the two sets is designed to hybridize to specific sequences (e.g., germline) in the target nucleic acids.

As mentioned, the present invention provides designed chip which contains a set of oligos that is derived from the germline gene segments and in addition contains oligos that have a single mutation at each one of the oligo bases (see Table 1). As the length of the oligo used is longer there is a greater need to insert more mutations. Since the variance of a certain antibody from the germline sequence is about 10%, when using 8 mers it is sufficient to use single mutations. However, if 12 mers are used than the oligo set should contain oligos with both a single mutation and oligos with two mutations. It should be noted that even if a certain 12 base sequence within the variable has more than 2 mutations, then the correct sequence might still be revealed, assuming that the three mutations are not juxtaposed (see Example 11E). The amount of mutation in the region of CDRs is higher than in the region of the frameworks and in addition the CDRs compose only ˜⅓ of the variable region. Therefore, the chip contains more variants of mutation for the region of the CDRs, such as 12 mers oligos that in the framework will be designed to contain 0-2 mutations whereas for the CDRs the oligos will be designed to contain 0-4 mutations.

Methods of producing probe arrays are well known in the art. State-of-the-art methods involves using a robotic apparatus to apply or “spot” distinct solutions containing probe molecules to closely spaced specific addressable locations on the surface of a planar support, typically a glass support, such as a microscope slide, which is subsequently processed by suitable thermal and/or chemical treatment to attach probe molecules to the surface of the support. Suitable supports may also include silicon, nitrocellulose, paper, cellulosic supports, and the like.

Ample guidance for obtaining and utilizing a probe array for suitably practicing the method of the present invention is provided in the literature of the art (for example, refer to: U.S. Pat. No. 6,551,784; U.S. Pat. No. 6,251,601; Forster et al., 2003. J. Endocrinol. 178:195-204; Howbrook et al., 2003. Drug Discov Today 8:642-51; Xiang et al., 2003. Curr Opin Drug Discov Devel. 6:384; Hardiman G., 2003. Pharmacogenomics 4:251). Custom designed arrays can be purchased from commercial suppliers [for example, Affymetrix, Santa Clara, USA; or Agilent Technologies, Palo Alto, USA).

As mentioned the oligonucleotide of the present invention are contacted with the population of polynucleotides under conditions which allow the formation of duplexes between at least a portion of the oligonucleotides and the population of polynucleotides.

Conditions which allow the formation of duplexes between at least a portion of the oligonucleotides and the population of polynucleotides include are described in Cowie (2004) infra.

To detect hybridization duplexes, either the target nucleic acids or probes (such as the soluble set of probes used in cSBH), are labeled with a detectable moiety (tag). Various types of detectable labels are known in the art. Preferably, the label is a fluorophore. Examples of fluorophores which can be used in the present invention include, but are not limited to, Cy5, Cy3, fluorescein isothiocyanate (FITC), phycoerythrin (PE), rhodamine, Texas red, and the like. Ample general guidance regarding fluorophore selection, and methods of conjugating a fluorophore to a polynucleotide is available in the literature of the art [refer, for example, to: Richard P. Haugland, “Molecular Probes: Handbook of Fluorescent Probes and Research Chemicals 1992-1994”, 5th ed., Molecular Probes, Inc. (1994); Hermanson, “Bioconjugate Techniques”, Academic Press New York, N.Y. (1995); Kay M. et al., 1995. Biochemistry 34:293; Stubbs et al., 1996. Biochemistry 35:937; U.S. Pat. No. 6,350,466 to Targesome, Inc.; U.S. Pat. No. 6,037,137 to Oncoimmunin Inc.]. For specific guidance regarding conjugating of a DNA molecule, with a fluorophore in the context of hybridization microarray applications, such as the method of the present invention, refer, for example, to Richter et al., 2002. Biotechniques 33(3):620.

Alternately, the label can be a radioactive atom (“radiolabel”; for example, 3-hydrogen, 125-iodine, 35-sulfur, 14-carbon, or 32-phosphorus), an enzyme which catalyzes a reaction resulting in a chromogenic substrate, (“enzymatic label”), colloidal gold, or any other suitable detectable label. For a detailed review of methods of conjugating a polynucleotide with a suitable detectable label for practicing the method of the present invention, and for detecting such labels in the context of the present invention, refer, for example, to Laboratory Techniques in Biochemistry and Molecular Biology, Vol. 24: Hybridization With Nucleic Acid Probes, P. Tijssen, ed. Elsevier, N.Y., (1993). Detectable labels suitable for use in the present invention include any composition detectable by spectroscopic, photochemical, biochemical, immunochemical, electrical, optical or chemical means. Patents teaching the use of suitable detectable labels include U.S. Pat. Nos. 3,817,837; 3,850,752; 3,939,350; 3,996,345; 4,277,437; 4,275,149; and 4,366,241.

Examples of suitable enzymatic detectable labels for practicing the method of the present invention include horseradish peroxidase (HRP) beta-galactosidase, and alkaline phosphatase (AP). Ample guidance for suitably obtaining and utilizing enzymatic detectable labels for practicing the method of the present invention is provided in the literature of the art (for example, refer to: Khatkhatay M I. and Desai M., 1999. J Immunoassay 20:151-83; Wisdom G B., 1994. Methods Mol. Biol. 32:433-40; Ishikawa E. et al., 1983. J Immunoassay 4:209-327; Oellerich M., 1980. J Clin Chem Clin Biochem. 18:197-208; Schuurs A H. and van Weemen B K., 1980. J Immunoassay 1:229-49).

Depending on the application and purpose, the nucleic acid target or soluble probes may be conjugated with the label during any of the various stages of the method of the present invention, and via any of various means well known to those of skill in the art.

Means of quantitavely detecting hybridization duplexes are well known in the art. See for example, Biochemistry and Molecular Biology, Vol. 24: Hybridization With Nucleic Acid Probes, P. Tijssen, ed. Elsevier, N.Y., (1993); and U.S. Pat. Nos. 3,817,837; 3,850,752; 3,939,350; 3,996,345; 4,277,437; 4,275,149; and 4,366,241]. For example, a fluorophore detectable label may be detected using a photodetector to detect emitted light, a radioactive detectable label may be detected using photographic film or a scintillation counter, an enzymatic detectable label may be detected by exposing the enzyme label to its substrate and detecting the reaction product produced by the action of the enzyme on the substrate, and a colloidal gold label may be detected by measuring light scattering thereby. Preferably, the pattern of array sites lighted-up (positively hybridizing) is analyzed with computer assistance to compile the set of sequences of the population of positively hybridizing oligonucleotides. This is effected by the following guidelines which are described in details in Example 14 of the Examples section.

(i) identifying oligonucleotides involved in the formation of the hybridization duplexes in a quantum above a predetermined threshold, so as to define a population of positively hybridizing oligonucleotides;

(ii) identifying germline sequences of germline segments in at least a portion of the positively hybridizing oligonucleotides or in at least a portion of the oligonucleotides involved in the formation of the hybridization complexes; Preferably selected are those oligonucleotides which hybridize to non-redundant (specific) germline sequences in the germline segment. Germline sequences identified herein also include germline sequences which include mutations. This is effected by identifying mutated oligos which exhibit higher score than non mutated germline oligos.

(iii) identifying positively hybridizing oligonucleotides overlapping with the germline sequences, thereby identifying germline junction sequences; and

(iv) assembling sequence information obtained from steps (ii) and (iii), thereby compiling the set of sequences of the population of polynucleotides;

Assembly process is initiated by the sequential knowledge of the germline genes and the way they recombine to produce the variable region i.e. VJ or VDJ. Essentially, the oligos are aligned using the above sequences as templates. Such an assembly process is similar to resequencing of known DNA sequences effected to detect sequence variations of single nucleotide polymorphisms [(SNPs), as reviewed by Hacia Nat. Genet. 1999 21(1 Suppl):42-7]. Though, in this case, sequence deviations contributing to the generation of variable regions (i.e., junctions) are much larger then single nucleotide changes necessitating the combination of re-sequencing along with de-novo sequencing.

A number of commonly used computer software fragment read assemblers capable of forming clusters of sequences are now available. These can be used provided that both oligo sequences as well as template sequences and arrangement thereof (i.e., VJ or VDJ) are provided as an input. These packages include but are not limited to, The TIGR Assembler [Sutton G. et al. (1995) Genome Science and Technology 1:9-19], GAP [Bonfield J K. et al. (1995) Nucleic Acids Res. 23:4992-4999], CAP2 [Huang X. et al. (1996) Genomics 33:21-31], The Genome Construction Manager [Laurence C B. Et al. (1994) Genomics 23:192-201], Bio Image Sequence Assembly Manager, SeqMan [Swindell S R. and Plasterer J N. (1997) Methods Mol. Biol. 70:75-89].

Once the sets of compiled sequences is at hand, the level thereof is determined (based on hybridization signal and preferably relative to normalization and expression controls, described above) to thereby quantify the expression of the population of polynucleotides encoding the antibodies or T cell receptors.

Sequence data and related information (e.g., level of expression) may be stored on a computer readable storage medium. The computer readable storage medium can further include information pertaining to generation of the data and/or potential uses therefor.

As used herein, a “computer-readable medium” refers to any medium that can be read and accessed directly by a machine [e.g., a digital or analog computer; e.g., a desktop PC, laptop, mainframe, server (e.g., a web server, network server, or server farm), a handheld digital assistant, pager, mobile telephone, or the like]. Computer-readable media include: magnetic storage media; such as floppy discs, hard disc storage medium, and magnetic tape; optical storage media such as CD-ROM; electrical storage media such as RAM, ROM, EPROM, EEPROM, flash memory, and the like; and hybrids of these categories such as magnetic/optical storage media.

A variety of data storage structures are available to those of ordinary skill in the art and can be used to create a computer-readable medium that has recorded one or more (or all) of the nucleic acids and/or amino acid sequences of the present invention. The data storage structure will generally depend on the means chosen to access the stored information. In addition, a variety of data processor programs and formats can be used to store the sequence information of the present invention on machine or computer-readable medium. The sequence information can be represented in a word processing text file, formatted in commercially-available software such as Microsoft Word, or represented in the form of an ASCII file, stored in a database application, such as DB2, Sybase, Oracle, or the like. One of ordinary skill in the art can readily adapt any number of data processor structuring formats (e.g., text file or database) to obtain machine or computer-readable medium having recorded thereon the sequence information of the present invention.

The above described sequence information and related information are stored in a relational database (such as Sybase or Oracle) that can have a first table for storing sequence information. The sequence information can be stored in one field (e.g., a first column) of a table row and an identifier for the sequence can be stored in another field (e.g., a second column) of the table row. The database can have a second table (to, for example, store related information, such as level of expression, immune status and associated diseases).

Once an antibody or TCR repertoire is associated with an antigen associated disease it can be used to identify protective antibodies as well as pathogenic antigens, which may be used to design therapeutic drugs for any antigen associated disease.

As used herein, the phrase “antigen associated disease” refers to any disease associated with a protective antigen specific immune response, potentially associated with a protective antigen specific immune response, or associated with a pathogenic antigen specific immune response.

As used herein, the term “disease” refers to any medical disease, disorder, condition, or syndrome, or to any undesired and/or abnormal physiological morphological, and/or physical state and/or condition.

Examples of autoimmune diseases associated with antibody mediated immune responses include, but are not limited to, rheumatoid diseases, rheumatoid autoimmune diseases, rheumatoid arthritis (Krenn V. et al., Histol Histopathol 2000 July; 15 (3):791), spondylitis, ankylosing spondylitis (Jan Voswinkel et al., Arthritis Res 2001; 3 (3): 189), systemic diseases, systemic autoimmune diseases, systemic lupus erythematosus (Erikson J. et al., Immunol Res 1998; 17 (1-2):49), sclerosis, systemic sclerosis (Renaudineau Y. et al., Clin Diagn Lab Immunol. 1999 March; 6 (2):156); Chan O T. et al., Immunol Rev 1999 June; 169:107), glandular diseases, glandular autoimmune diseases, pancreatic autoimmune diseases, diabetes, Type I diabetes (Zimmet P. Diabetes Res Clin Pract 1996 October; 34 Suppl:S125), thyroid diseases, autoimmune thyroid diseases, Graves' disease (Orgiazzi J. Endocrinol Metab Clin North Am 2000 June; 29 (2):339), thyroiditis, spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), myxedema, idiopathic myxedema (Mitsuma T. Nippon Rinsho. 1999 August; 57 (8):1759); autoimmune reproductive diseases, ovarian diseases, ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), repeated fetal loss (Tincani A. et al., Lupus 1998; 7 Suppl 2:S107-9), neurodegenerative diseases, neurological diseases, neurological autoimmune diseases, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83), motor neuropathies (Komberg A J. J Clin Neurosci. 2000 May; 7 (3):191), Guillain-Barre syndrome, neuropathies and autoimmune neuropathies (Kusunoki S. Am J Med Sci. 2000 April; 319 (4):234), myasthenic diseases, Lambert-Eaton myasthenic syndrome (Takamori M. Am J Med Sci. 2000 April; 319 (4):204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) 2000 January; 156 (1):23); neuropathies, dysimmune neuropathies (Nobile-Orazio E. et al., Electroencephalogr Clin Neurophysiol Suppl 1999; 50:419); neuromyotonia, acquired neuromyotonia, arthrogryposis multiplex congenita (Vincent A. et al., Ann N Y Acad. Sci. 1998 May 13; 841:482), cardiovascular diseases, cardiovascular autoimmune diseases, atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), granulomatosis, Wegener's granulomatosis, arteritis, Takayasu's arteritis and Kawasaki syndrome (Praprotnik S. et al., Wien Klin Wochenschr 2000 Aug. 25; 112 (15-16):660); anti-factor VIII autoimmune disease (Lacroix-Desmazes S. et al., Semin Thromb Hemost. 2000; 26 (2):157); vasculitises, necrotizing small vessel vasculitises, microscopic polyangitis, Churg and Strauss syndrome, glomerulonephritis, pauci-immune focal necrotizing glomerulonephritis, crescentic glomerulonephritis (Noel L H. Ann Med Interne (Paris). 2000 May; 151 (3):178); antiphospholipid syndrome (Flamholz R. et al., J Clin Apheresis 1999; 14 (4):171); heart failure, agonist-like beta-adrenoceptor antibodies in heart failure (Wallukat G. et al., Am J. Cardiol. 1999 Jun. 17; 83 (12A):75H), thrombocytopenic purpura (Moccia F. Ann Ital Med Int. 1999 April-June; 14 (2):114); hemolytic anemia, autoimmune hemolytic anemia (Efremov D G. et al., Leuk Lymphoma 1998 January; 28 (3-4):285), gastrointestinal diseases, autoimmune diseases of the gastrointestinal tract, intestinal diseases, chronic inflammatory intestinal disease (Garcia Herola A. et al., Gastroenterol Hepatol. 2000 January; 23 (1):16), celiac disease (Landau Y E. and Shoenfeld Y. Harefuah 2000 Jan. 16; 138 (2):122), autoimmune diseases of the musculature, myositis, autoimmune myositis, Sjogren's syndrome (Feist E. et al., Int Arch Allergy Immunol 2000 September; 123 (1):92); smooth muscle autoimmune disease (Zauli D. et al., Biomed Pharmacother 1999 June; 53 (5-6):234), hepatic diseases, hepatic autoimmune diseases, autoimmune hepatitis (Manns M P. J Hepatol 2000 August; 33 (2):326) and primary biliary cirrhosis (Strassburg C P. et al., Eur J Gastroenterol Hepatol. 1999 June; 11 (6):595).

Examples of diseases associated with T cell mediated autoimmune diseases, include, but are not limited to, rheumatoid diseases, rheumatoid arthritis (Tisch R, McDevitt HO. Proc Natl Acad Sci USA 1994 Jan. 18; 91 (2):437), systemic diseases, systemic autoimmune diseases, systemic lupus erythematosus (Datta S K., Lupus 1998; 7 (9):591), glandular diseases, glandular autoimmune diseases, pancreatic diseases, pancreatic autoimmune diseases, Type 1 diabetes (Castano L. and Eisenbarth G S. Ann. Rev. Immunol. 8:647); thyroid diseases, autoimmune thyroid diseases, Graves' disease (Sakata S. et al., Mol Cell Endocrinol 1993 March; 92 (1):77); ovarian diseases (Garza K M. et al., J Reprod Immunol 1998 February; 37 (2):87), prostatitis, autoimmune prostatitis (Alexander R B. et al., Urology 1997 December; 50 (6):893), polyglandular syndrome, autoimmune polyglandular syndrome, Type I autoimmune polyglandular syndrome (Hara T. et al., Blood. 1991 Mar. 1; 77 (5):1127), neurological diseases, autoimmune neurological diseases, multiple sclerosis, neuritis, optic neuritis (Soderstrom M. et al., J Neurol Neurosurg Psychiatry 1994 May; 57 (5):544), myasthenia gravis (Oshima M. et al., Eur J Immunol 1990 December; 20 (12):2563), stiff-man syndrome (Hiemstra H S. et al., Proc Natl Acad Sci USA 2001 Mar. 27; 98 (7):3988), cardiovascular diseases, cardiac autoimmunity in Chagas' disease (Cunha-Neto E. et al., J Clin Invest 1996 Oct. 15; 98 (8):1709), autoimmune thrombocytopenic purpura (Semple J W. et al., Blood 1996 May 15; 87 (10):4245), anti-helper T lymphocyte autoimmunity (Caporossi A P. et al., Viral Immunol 1998; 11 (1):9), hemolytic anemia (Sallah S. et al., Ann Hematol 1997 March; 74 (3): 139), hepatic diseases, hepatic autoimmune diseases, hepatitis, chronic active hepatitis (Franco A. et al., Clin Immunol Immunopathol 1990 March; 54 (3):382), biliary cirrhosis, primary biliary cirrhosis (Jones D E. Clin Sci (Colch) 1996 November; 91 (5):551), nephric diseases, nephric autoimmune diseases, nephritis, interstitial nephritis (Kelly C J. J Am Soc Nephrol 1990 August; 1(2):140), connective tissue diseases, ear diseases, autoimmune connective tissue diseases, autoimmune ear disease (Yoo T J. et al., Cell Immunol 1994 August; 157 (1):249), disease of the inner ear (Gloddek B. et al., Ann N Y Acad Sci 1997 Dec. 29; 830:266), skin diseases, cutaneous diseases, dermal diseases, bullous skin diseases, pemphigus vulgaris, bullous pemphigoid and pemphigus foliaceus.

Examples of antigen associated diseases associated with antigen specific delayed type hypersensitivity include, but are not limited to, contact dermatitis and drug eruption.

Examples of organ/tissue specific autoimmune diseases include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), Wegener's granulomatosis, Takayasu's arteritis, Kawasaki syndrome (Praprotnik S. et al., Wien Klin Wochenschr 2000 Aug. 25; 112 (15-16):660), anti-factor VIII autoimmune disease (Lacroix-Desmazes S. et al., Semin Thromb Hemost. 2000; 26 (2):157), necrotizing small vessel vasculitis, microscopic polyangiitis, Churg and Strauss syndrome, pauci-immune focal necrotizing and crescentic glomemlonephritis (Noel L H. Ann Med Interne (Paris). 2000 May; 151 (3):178), antiphospholipid syndrome (Flamholz R. et al., J Clin Apheresis 1999; 14 (4):171), antibody-induced heart failure (Wallukat G. et al., Am J. Cardiol. 1999 Jun. 17; 83 (12A):75H), thrombocytopenic purpura (Moccia F. Ann Ital Med Int. 1999 April-June; 14 (2):114; Semple J W. et al., Blood 1996 May 15; 87 (10):4245), autoimmune hemolytic anemia (Efremov D G. et al., Leuk Lymphoma 1998 January; 28 (3-4):285; Sallah S. et al., Ann Hematol 1997 March; 74 (3):139), cardiac autoimmunity in Chagas' disease (Cunha-Neto E. et al., J Clin Invest 1996 Oct. 15; 98 (8):1709) and anti-helper T lymphocyte autoimmunity (Caporossi A P. et al., Viral Immunol 1998; 11 (1):9).

Examples of autoimmune rheumatoid diseases include, but are not limited to rheumatoid arthritis (Krenn V. et al., Histol Histopathol 2000 July; 15 (3):791; Tisch R, McDevitt HO. Proc Natl Acad Sci units S A 1994 Jan. 18; 91 (2):437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. diseases include, but are not limited to autoimmune diseases of the pancreas, Type 1 diabetes (Castano L. and Eisenbarth G S. Arm. Rev. Immunol. 8:647; Zimmet P. Diabetes Res Clin Pract 1996 October; 34 Suppl:S125), autoimmune thyroid diseases, Graves' disease (Orgiazzi J. Endocrinol Metab Clin North Am 2000 June; 29 (2):339; Sakata S. et al., Mol Cell Endocrinol 1993 March; 92 (1):77), spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), idiopathic myxedema (Mitsuma T. Nippon Rinsho. 1999 August; 57 (8):1759), ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), autoimmune prostatitis (Alexander R B. et al., Urology 1997 December; 50 (6):893) and Type I autoimmune polyglandular syndrome (Hara T. et al., Blood. 1991 Mar. 1; 77 (5):1127).

Examples of autoimmune gastrointestinal diseases include, but are not limited to, chronic inflammatory intestinal diseases (Garcia Herola A. et al., Gastroenterol Hepatol. 2000 January; 23 (1):16), celiac disease (Landau Y E. and Shoenfeld Y. Harefuah 2000 Jan. 16; 138 (2):122), colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis (Franco A. et al., Clin Immunol Immunopathol 1990 March; 54 (3):382), primary biliary cirrhosis (Jones D E. Clin Sci (Colch) 1996 November; 91 (5):551; Strassburg C P. et al., Eur J Gastroenterol Hepatol. 1999 June; 11 (6):595) and autoimmune hepatitis (Manns M P. J Hepatol 2000 August; 33 (2):326).

Examples of autoimmune neurological diseases include, but are not limited to, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83; Oshima M. et al., Eur J Immunol 1990 December; 20 (12):2563), neuropathies, motor neuropathies (Kornberg A J. J Clin Neurosci. 2000 May; 7 (3):191); Guillain-Barre syndrome and autoimmune neuropathies (Kusunoki S. Am J Med Sci. 2000 April; 319 (4):234), myasthenia, Lambert-Eaton myasthenic syndrome (Takamori M. Am J Med Sci. 2000 April; 319 (4):204); paraneoplastic neurological diseases, cerebellar atrophy, paraneoplastic cerebellar atrophy and stiff-man syndrome (Hiemstra H S. et al., Proc Natl Acad Sci units S A 2001 Mar. 27; 98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 January; 156 (1):23); dysimmune neuropathies (Nobile-Orazio E. et al., Electroencephalogr Clin Neurophysiol Suppl 1999; 50:419); acquired neuromyotonia, arthrogryposis multiplex congenita (Vincent A. et al., Ann N Y Acad. Sci. 1998 May 13; 841:482), neuritis, optic neuritis (Soderstrom M. et al., J Neurol Neurosurg Psychiatry 1994 May; 57 (5):544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 September; 123 (1):92) and smooth muscle autoimmune disease (Zauli D. et al., Biomed Pharmacother 1999 June; 53 (5-6):234).

Examples of autoimmune nephric diseases include, but are not limited to, nephritis and autoimmune interstitial nephritis (Kelly C J. J Am Soc Nephrol 1990 August; 1 (2):140).

Examples of autoimmune diseases related to reproduction include, but are not limited to, repeated fetal loss (Tincani A. et al., Lupus 1998; 7 Suppl 2:S107-9).

Examples of autoimmune connective tissue diseases include, but are not limited to, ear diseases, autoimmune ear diseases (Yoo T J. et al., Cell Immunol 1994 August; 157 (1):249) and autoimmune diseases of the inner ear (Gloddek B. et al., Ann N Y Acad Sci 1997 Dec. 29; 830:266).

Examples of autoimmune systemic diseases include, but are not limited to, systemic lupus erythematosus (Erikson J. et al., Immunol Res 1998; 17 (1-2):49) and systemic sclerosis (Renaudineau Y. et al., Clin Diagn Lab Immunol. 1999 March; 6 (2):156); Chan O T. et al., Immunol Rev 1999 June; 169:107).

Examples of antigen specific infectious diseases include, but are not limited to, chronic infectious diseases, subacute infectious diseases, acute infectious diseases, viral diseases, bacterial diseases, protozoan diseases, parasitic diseases, fungal diseases, mycoplasma diseases and prion diseases.

Examples of antigen specific transplantation related diseases, but are not limited to, graft rejection, chronic graft rejection, subacute graft rejection, hyperacute graft rejection, acute graft rejection and graft versus host disease.

Examples of allergic diseases include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

Examples of antigen specific inflammatory diseases include, but are not limited to; inflammation associated with injuries, neurodegenerative diseases, ulcers, prosthetic implants, menstruation, septic shock, anaphylactic shock, toxic shock syndrome, cachexia, necrosis and gangrene; musculo-skeletal inflammations, idiopathic inflammations.

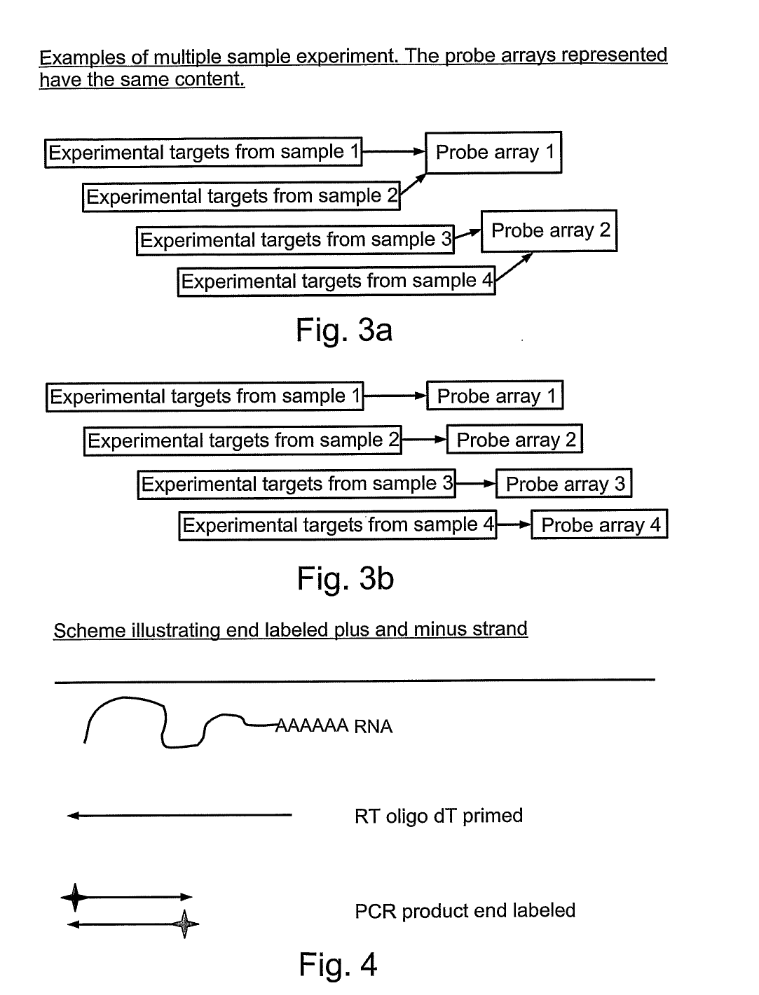

Crossing this information retrieved by the teachings of the present invention described above among multiple samples and populations can reveal important novel information pertaining to antibodies and pathogenic antigens along with correlation to development, health condition (see FIG. 3).

1. Oligos Profile

This output reveals the individual oligos hybridization intensity as revealed after preprocessing steps. This output can be used in analysis involving multiple samples in order to find similarities and/or differences.

2. Germline Profile

This output reveals the separate germline gene fragments expression level. The junctions are not evident in this output.

3. Complete Chain Profile

This output reveals the complete variable regions existing within the sample with their relative amounts. This output contains both the nucleotide and the protein sequence information. The protein is determined by the translation of the nucleotide sequences. In addition it can identify nucleotide sequences that can produce a fully active antibody.

The present invention provides unique advantages compared to other methods known in the art:

1. High Throughput: this method allows analysis of a large amount of cells and samples in a fast manner in order to identify a number of relevant target nucleic acids.

Analyze a mixture of a plurality of nucleic acids: this method allows simultaneous analysis of a plurality of nucleic acids, meaning it is not restricted to analysis of a single target molecule.

2. Multiple population analysis: ability to easily screen a broad spectrum of samples, considering the disease stage, developmental stage, type of treatment and other measures. This supports the ability to identify a number of relevant targets of greatest effectiveness prospects.

3. Analyze the quantitative level of expression of the various target nucleic acids: this ability is achieved by analyzing the extent of hybridization to multiple probes defining the target nucleic acids. There is evidence showing that the V family usage is restricted and therefore a mixture of targets found within a sample can have a commonality or in other words they can have a restricted repertoire. Furthermore, this repertoire can be significantly different from a second sample

4. Integrated discovery of novel targets: ability to identify their sequence or partial sequence and/or classify the target gene as well as their expression profile. This allows concentrating on targets with a significant profile, meaning higher correlation with population sub-type and ability to scan for the more pronounced targets.

5. Ability to discover human biological targets. The advantages of the “true” targets are that they have undergone selective pressure that: restricts them from identifying self and insures high affinity and specific binding to antigen.

6. Shortening of target discovery and antibody manufacture time: Currently examining the repertoire of target genes in a diverse sample requires cloning of the PCR products and then individual clone picking and sequencing which is labor and time demanding (a few days). The extent of clone picking and sequencing is proportional to the amount of complexity within a sample. However, this information is not known a priori. In addition in order to infer on the expression level of a certain sequence within the sample one needs to perform massive analysis. These steps are shortened to a days work using this application method.

7. Ability to simultaneously explore both TCR and Ig genes: Information on both these target types within the same sample can result in a more comprehensive picture of the immune response.

8. Obtaining a signature or profile of the target genes within populations: The amount of hybridization between the targets in the sample and the probe set taken as a whole composes a profile or signature. The study of populations profile can reveal their environmental, disease or any other influencing factor history. This information could be used for diagnostic and research purposes.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

The following examples are to be considered merely as illustrative and non-limiting in nature. It will be apparent to one skilled in the art to which the present invention pertains that many modifications, permutations, and variations may be made without departing from the scope of the invention.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W.H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

TABLE 1

All Possible Oligos Derived from Human
Gene Fragment VH1-18 (Base 1 to 12)
Containing Zero-OneMutations

| | Start | # Mutations | SEQ | |
|---|---|---|---|---|
| Gene | position | (position in | ID | |
| fragment | in gene | oligo,type) | NO: | Oligo sequence |
| VH1-18 | 1 | 0 | 19 | CAGGTTCAGCTG |
| VH1-18 | 1 | 1 (1, c -> g) | 20 | gAGGTTCAGCTG |
| VH1-18 | 1 | 1 (1, c -> a) | 21 | aAGGTTCAGCTG |
| VH1-18 | 1 | 1 (1, c -> t) | 22 | tAGGTTCAGCTG |
| VH1-18 | 1 | 1 (2, a -> c) | 23 | CcGGTTCAGCTG |
| VH1-18 | 1 | 1 (2, a -> g) | 24 | CgGGTTCAGCTG |
| VH1-18 | 1 | 1 (2, a -> t) | 25 | CtGGTTCAGCTG |
| VH1-18 | 1 | 1 (3, g -> e) | 26 | CAcGTTCAGCTG |
| VH1-18 | 1 | 1 (3, g -> a) | 27 | CAaGTTCAGCTG |
| VH1-18 | 1 | 1 (3, g -> t) | 28 | CAtGTTCAGCTG |
| VH1-18 | 1 | 1 (4, g -> c) | 29 | CAGcTTCAGCTG |
| VH1-18 | 1 | 1 (4, g -> a) | 30 | CAGaTTCAGCTG |
| VH1-18 | 1 | 1 (4, g -> t) | 31 | CAGtTTCAGCTG |
| VH1-18 | 1 | 1 (5, t -> a) | 32 | CAGGaTCAGCTG |
| VH1-18 | 1 | 1 (5, t -> c) | 33 | CAGGcTCAGCTG |
| VH1-18 | 1 | 1 (5, t -> g) | 34 | CAGGgTCAGCTG |
| VH1-18 | 1 | 1 (6, t -> a) | 35 | CAGGTaCAGCTG |
| VH1-18 | 1 | 1 (6, t -> c) | 36 | CAGGTcCAGCTG |
| VH1-18 | 1 | 1 (6, t -> g) | 37 | CAGGTgCAGCTG |
| VH1-18 | 1 | | | |
| VH1-18 | 1 | 1 (7, c -> a) | 38 | CAGGTTaAGCTG |
| VH1-18 | 1 | 1 (7, c -> t) | 39 | CAGGTTtAGCTG |
| VH1-18 | 1 | 1 (7, c -> g) | 40 | CAGGTTgAGCTG |
| VH1-18 | 1 | 1 (8, a -> t) | 41 | CAGGTTCtGCTG |
| VH1-18 | 1 | 1 (8, a -> c) | 42 | CAGGTTCcGCTG |
| VH1-18 | 1 | 1 (8, a -> g) | 43 | CAGGTTCgGCTG |
| VH1-18 | 1 | 1 (9, g -> c) | 44 | CAGGTTCAcCTG |
| VH1-18 | 1 | 1 (9, g -> a) | 45 | CAGGTTCAaCTG |
| VH1-18 | 1 | 1 (9, g -> t) | 46 | CAGGTTCAtCTG |
| VH1-18 | 1 | 1 (10, c -> g) | 47 | CAGGTTCAGgTG |
| VH1-18 | 1 | 1 (10, c -> a) | 48 | CAGGTTCAGaTG |
| VH1-18 | 1 | 1 (10, c -> t) | 49 | CAGGTTCAGtTG |
| VH1-18 | 1 | 1 (11, t -> a) | 50 | CAGGTTCAGCaG |
| VH1-18 | 1 | 1 (11, t -> c) | 51 | CAGGTTCAGCcG |
| VH1-18 | 1 | 1 (11, t -> g) | 52 | CAGGTTCAGCgG |
| VH1-18 | 1 | 1 (12, g -> a) | 53 | CAGGTTCAGCTa |
| VH1-18 | 1 | 1 (12, g -> c) | 54 | CAGGTTCAGCTc |
| VH1-18 | 1 | 1 (12, g -> t) | 55 | CAGGTTCAGCTt |

Mutated bases are indicated by small letters.

### Example 2

TABLE 2
Table 2: Representation of the Ig Heavy Chain Gene Segments
One variable germline gene fragment (out of the 51 genes)
recombines with one D (out of 27) and then with one J gene
segment (out of 6). N (0-15) random bases are inserted
in the junctions between the segments. The gene fragments
indicated by bold represent a certain recombination event
joining VH1, 1-03 with D2, 2-21 and JH4 (V-(N)-D-(N)-J).

| | Sub | | Sub | | Sub |
|---|---|---|---|---|---|
| # | Family | Locus | Family | Locus | Family |
| 1 | VH1 | 1-02 | D1 | 1-1 | JH1 |
| 2 | VH1 | 1-03 | D1 | 1-7 | JH2 |
| 3 | VH1 | 1-08 | D1 | 1-14 | JH3 |
| 4 | VH1 | 1-18 | D1 | 1-20 | JH4 |
| 5 | VH1 | 1-24 | D1 | 1-26 | JH5 |
| 6 | VH1 | 1-45 | D2 | 2-2 | JH6 |
| 7 | VH1 | 1-46 | D2 | 2-8 | |
| 8 | VH1 | 1-58 | D2 | 2-15 | |
| 9 | VH1 | 1-69 | D2 | 2-21 | |
| 10 | VH1 | 1-e | D3 | 3-3 | |
| 11 | VH1 | 1-f | D3 | 3-9 | |
| 12 | VH2 | 2-05 | D3 | 3-10 | |
| 13 | VH2 | 2-26 | D3 | 3-16 | |
| 14 | VH2 | 2-70 | D3 | 3-22 | |
| 15 | VH3 | 3-07 | D4 | 4-4 | |
| 16 | VH3 | 3-09 | D4 | 4-11 | |
| 17 | VH3 | 3-11 | D4 | 4-17 | |
| 18 | VH3 | 3-13 | D4 | 4-23 | |
| 19 | VH3 | 3-15 | D5 | 5-5 | |
| 20 | VH3 | 3-20 | D5 | 5-12 | |
| 21 | VH3 | 3-21 | D5 | 5-18 | |
| 22 | VH3 | 3-23 | D5 | 5-24 | |
| 23 | VH3 | 3-30 | D6 | 6-6 | |
| 24 | VH3 | 3-30.3 | D6 | 6-13 | |
| 25 | VH3 | 3-30.5 | D6 | 6-19 | |
| 26 | VH3 | 3-33 | D6 | 6-25 | |
| 27 | VH3 | 3-43 | D7 | 7-27 | |
| 28 | VH3 | 3-48 | | | |
| 29 | VH3 | 3-49 | | | |
| 30 | VH3 | 3-53 | | | |
| 31 | VH3 | 3-64 | | | |
| 32 | VH3 | 3-66 | | | |
| 33 | VH3 | 3-72 | | | |
| 34 | VH3 | 3-73 | | | |
| 35 | VH3 | 3-74 | | | |
| 36 | VH3 | 3-d | | | |
| 37 | VH3 | 4-04 | | | |
| 38 | VH4 | 4-28 | | | |
| 39 | VH4 | 4-30.1 | | | |
| 40 | VH4 | 4-30.2 | | | |
| 41 | VH4 | 4-30.4 | | | |
| 42 | VH4 | 4-31 | | | |
| 43 | VH4 | 4-34 | | | |
| 44 | VH4 | 4-39 | | | |
| 45 | VH4 | 4-59 | | | |
| 46 | VH4 | 4-61 | | | |
| 47 | VH4 | 4-b | | | |
| 48 | VH5 | 5-51 | | | |
| 49 | VH5 | 5-a | | | |
| 50 | VH6 | 6-01 | | | |
| 51 | VH7 | 7-4.1 | | | |

### Example 3

TABLE 3
Scoring the Various Sequence Assembly Options

| | SEQ | Score | Score |
|---|---|---|---|
| | ID | Data | Data |
| Sequence | NO: | 1 | 2 |

| | | | | | |
|---|---|---|---|---|---|
| a | VH4-61 | CTCCGTCAGCAGTGGTGGTTACTA | 56 | 10 | 10 |
| | (base | CTGGAGC | | | |
| | 81-111) | | | | |
| b | | CTCCATCAGCAGTAGTAGTTACTAC | 57 | 100 30 | |
| | | TGGGGC | | | |
| c | | CTCCGTCAGCAGTAGTAGTTACTA | 58 | 30 | 100 |
| | | CTGGAGC | | | |

The example in Table 3 above illustrates how to decide which of the sequences coexist in the sample. If the triple mutation (mutation 1 and 2) scores higher than the double one then it is mostly likely that it is the only predominate one (Data 1)., However, if the double mutation scores higher than it is most likely to be the only predominate one (Data 2). Using oligos of length 10 bases and higher will result in better discrimination.

### Example 4

TABLE 4
An Example for the Percent Expression of the
Germline Gene Fragments of a Sample

| | Sub | | % | Sub | | % | Sub | % |
|---|---|---|---|---|---|---|---|---|
| # | Family | Locus | Expression | Family | Locus | Expression | Family | Expression |
| 1 | VH1 | 1-02 | | D1 | 1-1 | | JH1 | |
| 2 | VH1 | 1-03 | | D1 | 1-7 | 50 | JH2 | 40 |
| 3 | VH1 | 1-08 | | D1 | 1-20 | | JH3 | |
| 4 | VH1 | 1-18 | | D1 | 1-26 | | JH4 | 60 |
| 5 | VH1 | 1-24 | | D2 | 2-2 | 10 | JH5 | |
| 6 | VH1 | 1-45 | 40 | D2 | 2-8 | | | |
| 7 | VH1 | 1-46 | | D2 | 2-15 | | | |
| 8 | VH1 | 1-58 | | D2 | 2-21 | 40 | | |
| 9 | VH1 | 1-69 | | D3 | 3-3 | | | |
| 10 | VH1 | 1-e | | D3 | 3-9 | | | |
| 11 | VH1 | 1-f | | D3 | 3-10 | | | |
| 12 | VH2 | 2-05 | | D3 | 3-16 | | | |
| 13 | VH2 | 2-26 | | D3 | 3-22 | | | |
| 14 | VH2 | 2-70 | | D4 | 4-4 | | | |
| 15 | VH3 | 3-07 | 10 | D4 | 4-11 | | | |
| 16 | VH3 | 3-09 | | D4 | 4-17 | | | |
| 17 | VH3 | 3-11 | | D4 | 4-23 | | | |
| 18 | VH3 | 3-13 | | D5 | 5-5 | | | |
| 19 | VH3 | 3-15 | | D5 | 5-12 | | | |
| 20 | VH3 | 3-20 | | D5 | 5-18 | | | |
| 21 | VH3 | 3-21 | | D5 | 5-24 | | | |
| 22 | VH3 | 3-23 | | D6 | 6-6 | | | |
| 23 | VH3 | 3-30 | | D6 | 6-13 | | | |
| 24 | VH3 | 3-30.3 | | | | | | |
| 25 | VH3 | 3-30.5 | | | | | | |
| 26 | VH3 | 3-33 | | | | | | |
| 27 | VH3 | 3-43 | | | | | | |
| 28 | VH3 | 3-48 | | | | | | |
| 29 | VH3 | 3-49 | | | | | | |
| 30 | VH3 | 3-53 | | | | | | |
| 31 | VH3 | 3-64 | | | | | | |
| 32 | VH3 | 3-66 | | | | | | |
| 33 | VH3 | 3-72 | | | | | | |
| 34 | VH3 | 3-73 | | | | | | |
| 35 | VH3 | 3-74 | | | | | | |
| 36 | VH3 | 3-d | | | | | | |
| 37 | VH4 | 4-04 | | | | | | |
| 38 | VH4 | 4-28 | | | | | | |
| 39 | VH4 | 4-30.1 | | | | | | |
| 40 | VH4 | 4-30.2 | | | | | | |
| 41 | VH4 | 4-30.4 | | | | | | |
| 42 | VH4 | 4-31 | | | | | | |
| 43 | VH4 | 4-34 | 50 | | | | | |
| 44 | VH4 | 4-39 | | | | | | |
| 45 | VH4 | 4-59 | | | | | | |
| 46 | VH4 | 4-61 | | | | | | |
| 47 | VH4 | 4-b | | | | | | |
| 48 | VH5 | 5-51 | | | | | | |
| 49 | VH5 | 5-a | | | | | | |
| 50 | VH6 | 6-01 | | | | | | |
| 51 | VH7 | 7-4.1 | | | | | | |

### Example 5

Oligos Designed for the V-D Junction.

FIG. 7 shows an example of the oligos selected to span the junction between VH7-4.1 (5′ sequence-TGTGCGAGAGA, SEQ ID NO: 60 X62110) and D1-1 (5′ sequence-GGTACAACTGGAACGAC, SEQ ID NO: 61 X97051). N represents all the bases possible (ATGC). In other words, there was a need to design oligos for all the possibilities designated by N (4096 combinations). If only one base is inserted then the best signals will be from the top and bottom oligos (below). However, if two bases were inserted then the best signals will be from the first and second top and bottom oligos.

### Example 6

RNA and/or DNA Extraction

DNA can be extracted from the sample according to means well known in the art (see, e.g., Maniatis, et al., Molecular Cloning; A Laboratory Manual (Cold Spring Harbor Lab, New York, 1982) or by using the RNA and/or DNA are extracted by commercial kits using the manufacturer's instructions (Amersham Pharmacia Biotech Ltd., Little Chalfont, UK) or by any other known in the art.

### Example 7

cDNA Synthesis and PCR Amplification

It has shown by numerous studies that the region of the variable region of TCR and antibodies can be amplified using specific primers for RT-PCR and PCR. The amplification is by using sets of oligonucleotide primers that can primer all possible chain sequences. The oligonucleotide sequences of the 5′ primers that are typically used are based on the N-terminal sequences of antibodies are the first framework determining region or the signal sequence. The sequences of the 3′ primers are based on the conserved regions of the first constant domain of antibodies or on the antibody J region sequences. A human Ig set of primers is available from Novagen. PCR is performed according to manufacture's protocol (Novagen, Germany).

### Example 8

Probe Labeling Procedures

a) PCR with end labeled fluorescent dye primers. The incorporation of the dye will be during the primer-annealing step. The 5′ primers (for instance Cy3 labeled) will complement the leader, FR1, FR2, and FR3. This should be a mixture of primers that will anneal to all rearranged genes at a defined position within the gene. The 3′ primer (for instance Cy5 labeled) is from the end of: FR2, FR3, FR4 (J region) or the beginning of the constant region. (see FIG. 4)

b) PCR as described above, with non-labeled primer. Instead, the labeling is done by performing the PCR in the presence of labeled nucleotides.

c) In either case the above procedures can be done separately for each chain type and chain subtype. Or different type of labels can be used for each of the chains types.

End labeling of oligonucleotides is performed by using the 3′ End Labeling Kit (NEN life Science Products, MA).

### Example 9

Probe Set (Chip Design).

The chip is composed of oligos in the range of 8-40 bases. Table 5 illustrates the number of overlapping oligos (with a single base shift) that are needed to cover the ˜40,000 bases of the human germline gene fragments.

TABLE 5
Statistics on the Oligo Frequency within the Ig Human
Germline Gene Fragments

| | # of different oligos | | |
|---|---|---|---|
| oligo length | spaning germline | matching more | |
| (bases) | gene fragments* | than 100 times | unique |
| 6 | 3468 | 11 | 594 |
| 7 | 7427 | 3 | 2706 |
| 9 | 13529 | 0 | 7750 |
| 11 | 16200 | 0 | 10281 |
| 13 | 17739 | 0 | 11754 |

*If n is the base length of an oligo then what is calculated is the number of different oligos needed in order to span all germline genes with n − 1 base overlap between the oligos. These oligos match the plus strand meaning that the target hybridizing is the minus strand. The oligos numbers calculated in the table do not take into consideration the junction sequence that composes the recombinant variable region.

It is evident that as the oligo length increases there is a decrease in the chance that a certain oligo sequence will occur more than once in a gene fragment or in more than a single germline gene type.

### Example 10

Determining Labeled Probe Level

After the chemical part of the experiment, it is subjected to image analysis. Data are collected by the scanner, digitized and stored. The task of the image processing hardware and software is to locate spots on the slide and to segment them, that is, to separate into the pixels bearing the signal and the pixels belonging to the background. There are several methods to perform this task as described in Holloway et al., Nat Genet suppl: 481-489, 2002 and Forster et al., J Endocrinol 178: 195-204, 2003.

### Example 11

Sequence Assembly Process

A. gi|23320665|gb|AY056842.1| Homo sapiens anti-HIV-1 gp120 immunoglobulin heavy chain variable region mRNA, partial cds [FIG. 8—Frameworks regions are underlined (FR1-FR4); CDR regions are shown in bold (CDR1-3)].

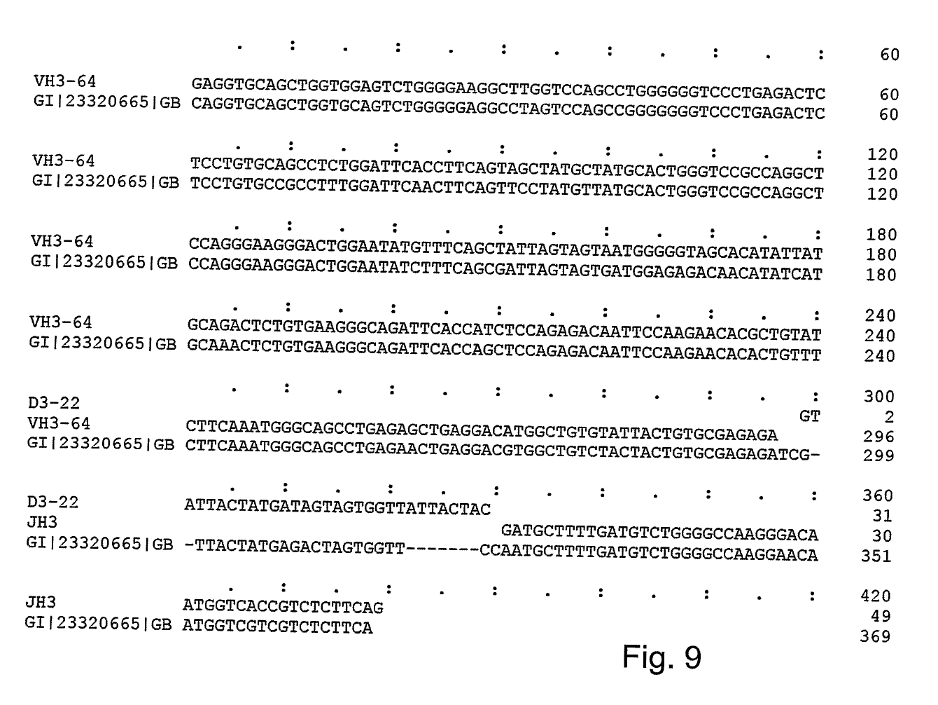

B. Multiple alignment of AY056842.1 with: VH3-64, D3-22 and JH3 is shown in FIG. 9.

C. Sequence from base 2-60 of AY056842.1 (framework 1).

Table 6 demonstrates the oligos from which the sequence of AY056842.1 was assembled when using a set of 12 mer oligos that have 0-2 mismatches with VH3-64 germline segment and the reverse complement (minus) strand of AY056842.1 as the target.

A match between AY056842.1 to VH3-64 is represented by a dot.

TABLE 6

| | | | | |
|---|---|---|---|---|
| (oligo | | # Mutations | | |
| name/SEQ | Position | (position in | SEQ | |
| ID NO: | in gene | oligo, type) | ID: | Sequence |

| | | | | |
|---|---|---|---|---|
| Germline- | Feb-60 | | | ..............A.........A....T.G........T.................. |
| VH3-64 | | | | |
| AY056842.1 | Feb-60 | | 62 | AGGTGCAGCTGGTGCAGTCTGGGGGAGGCCTAGTCCAGCCGGGGGGGTCCCTGAGACTC |
| VH3-64 | Feb-13 | 0 | 63 | AGGTGCAGCTGG |
| VH3-64 | Mar-14 | 0 | 64 | GGTGCAGCTGGT |
| VH3-64 | Apr-15 | 0 | 65 | GTGCAGCTGGTG |
| VH3-64 | May-16 | 1 (12, G -> C) | 66 | TGCAGCTGGTGC |
| VH3-64 | Jun-17 | 1 (11, G -> C) | 67 | GCAGCTGGTGCA |
| VH3-64 | Jul-18 | 1 (10, G -> C) | 68 | CAGCTGGTGCAG |
| VH3-64 | Aug-19 | 1 (9, G -> C) | 69 | AGCTGGTGCAGT |
| VH3-64 | Sep-20 | 1 (8, G -> C) | 70 | GCTGGTGCAGTC |
| VH3-64 | Oct-21 | 1 (7, G -> C) | 71 | CTGGTGCAGTCT |
| VH3-64 | Nov-22 | 1 (6, G -> C) | 72 | TGGTGCAGTCTG |
| VH3-64 | Dec-23 | 1 (5, G -> C) | 73 | GGTGCAGTCTGG |
| VH3-64 | 13-24 | 1 (4, G -> C) | 74 | GTGCAGTCTGGG |
| VH3-64 | 14-25 | 1 (3, G -> C) | 75 | TGCAGTCTGGGG |
| VH3-64 | 15-26 | 2 (2, G -> C); | 76 | GCAGTCTGGGGG |
| | | (12, A -> G) | | |
| VH3-64 | 16-27 | 2 (1, G -> C); | 77 | CAGTCTGGGGGA |
| | | (11, A -> G) | | |
| VH3-64 | 17-28 | 1 (10, A -> G) | 78 | AGTCTGGGGGAG |
| VH3-64 | 18-29 | 1 (9, A -> G) | 79 | GTCTGGGGGAGG |
| VH3-64 | 19-30 | 1 (8 ,A -> G) | 80 | TCTGGGGGAGGC |
| VH3-64 | 20-31 | 2 (7 ,A -> G); | 81 | CTGGGGGAGGCC |
| | | (12, T -> C) | | |
| VH3-64 | 21-32 | 2 (9,A -> G); | 82 | TGGGGGAGGCCT |
| | | (12, T -> C) | | |
| VH3-64 | 22-33 | 3 | 83 | GGGGGAGGCCTA * |
| VH3-64 | 23-34 | 3 | 84 | GGGGAGGCCTAG * |
| VH3-64 | 24-35 | 3 | 85 | GGGAGGCCTAGT * |
| VH3-84 | 25-36 | 3 | 86 | GGAGGCCTAGTC * |
| VH3-64 | 26-37 | 3 | 87 | GAGGCCTAGTCC * |
| VH3-64 | 27-38 | 2 (5,T -> C); | 88 | AGGCCTAGTCCA |
| | | (8, G -> A) | | |
| VH3-64 | 28-39 | 2 (4, T -> C); | 89 | GGCCTAGTCCAG |
| | | (7, -> A) | | |
| VH3-64 | 29-40 | 2 (3,T -> C); | 90 | GCCTAGTCCAGC |
| | | (6, G -> A) | | |
| VH3-64 | 30-41 | 2 (2, T -> C); | 91 | CCTAGTCCAGCC |
| | | (5, G -> A) | | |
| VH3-64 | 31-42 | 3 | 92 | CTAGTCCAGCCG * |
| VH3-64 | 32-43 | 2 (2, G -> A); | 93 | TAGTCCAGCCGG |
| | | (11, T -> 6) | | |
| VH3-64 | 33-44 | 2 (1, G -> A); | 94 | AGTCCAGCCGGG |
| | | (10, T -> 6) | | |
| VH3-64 | 34-45 | 1 (9, T -> 6) | 95 | GTCCAGCCGGGG |
| VH3-64 | 35-46 | 1 (8, T -> G) | 96 | TCCAGCCGGGGG |
| VH3-64 | 36-47 | 1 (7, T -> G) | 97 | CCAGCCGGGGGG |
| VH3-64 | 37-48 | 1 (6, T -> G) | 98 | CAGCCGGGGGGG |
| VH3-64 | 38-49 | 1 (5, T -> 6) | 99 | AGCCGGGGGGGT |
| VH3-64 | 39-50 | 1 (4, T -> G) | 100 | GCCGGGGGGGTC |
| VH3-64 | 40-51 | 1 (3, T -> G) | 101 | CCGGGGGGGTCC |
| VH3-64 | 41-52 | 1 (2, T -> G) | 102 | CGGGGGGGTCCC |
| VH3-64 | 42-53 | 1 (1, T -> G) | 103 | GGGGGGGTCCCT |
| VH3-64 | 43-54 | 0 | 104 | GGGGGGTCCCTG |
| VH3-64 | 44-55 | 0 | 105 | GGGGGTCCCTGA |
| VH3-64 | 45-56 | 0 | 106 | GGGGTCCCTGAG |
| VH3-64 | 46-57 | 0 | 107 | GGGTCCCTGAGA |
| VH3-64 | 47-58 | 0 | 108 | GGTCCCTGAGAC |
| VH3-64 | 48-59 | 0 | 109 | GTCCCTGAGACT |
| VH3-64 | 49-60 | 0 | 110 | TCCCTGAGACTC |

* represents oligos with three mutations. These oligos may not be present on the chip if the maximum number of designed mutations is two. However, the sequence might still be assembled as shown in section E.

D. Oligos that match the germline gene VH2-64. Two regions of the sequence are assembled using the oligos that completely match germline gene VH3-64.

TABLE 7

| | |
|---|---|
| SEQ | |
| ID: | ..............G.........A....T.G........T.................. |

| | |
|---|---|
| 62 | AGGTGCAGCTGGTGCAGTCTGGGGGAGGCCTAGTCCAGCCGGGGGGGTCCCTGAGACTC |
| 63 | AGGTGCAGCTGG |
| 64 | GGTGCAGCTGGT |
| 65 | GTGCAGCTGGTG |
| 104 | GGGGGGTCCCTG |
| 105 | GGGGGTCCCTGA |
| 106 | GGGGTCCCTGAG |
| 107 | GGGTCCCTGAGA |
| 108 | GGTCCCTGAGAC |
| 109 | GTCCCTGAGACT |
| 110 | TCCCTGAGACTC |

E. Some oligos in the overlapping scheme will not appear if the mutations designed will contain a maximum of two mutations. However, there is enough overlap to allow the complete sequence assembly.

TABLE 8

| Sequence | | # Mutations | | |
|---|---|---|---|---|
| (probe) | Position | (position in | SEQ | |
| name | in gene | (oligo, type) | ID: | Sequence |
| Germline | Jan-59 | | | ..............G.........A....T.G........T.................. |
| VH3-64 | | | | |
| AY056842.1 | Feb-60 | | 59 | AGGTGCAGCTGGTGCAGTCTGGGGGAGGCCTAGTCCAGCCGGGGGGGTCCCTGAGACTC |
| VH3-64 | 20-31 | 2 (9, A -> G): | 82 | TGGGGGAGGCCT |
| | | (12, T -> C) | | |
| VH3-64 | 26-37 | 2 (5, T -> C): | 88 | AGGCCTAGTCCA |
| | | (8, G ->A) | | |

F. Illustrated is the VDJ junction of sequence AY056842.1. All the oligos that contribute to its assembly are shown. Two types of oligos were used to construct the sequence 12 mers matching VH3-64, D3-22 and JH3 with 0-2 mutations and a set of all possible 8 mers.

TABLE 9a
continued in Table 9b

| | | # |
|---|---|---|
| | | Mutations |
| Sequence | | (position |
| (oligo) | Position | in |
| name | in gene | oligo, type) |
| AY056842.1 | 276 | |
| VH3-64 | 276 | |
| D3-22 | 1 | |
| JH3 | 1 | |
| VH3-64 | 276-287 | 2(4, G-> |
| | | C); (7, T |
| | | ->C) |
| VH3-64 | 277-288 | 2(3, G-> |
| | | C); (6, T |
| | | ->C) |
| VH3-64 | 278-289 | 2(2, G-> |
| | | C); (5, T |
| | | ->C) |
| VH3-64 | 279-290 | 2(1, G-> |
| | | C); (4, T |
| | | ->C) |
| VH3-64 | 280-291 | 1(3, T -> |
| | | C) |
| VH3-64 | 281-292 | 1(2, T -> |
| | | C) |
| VH3-64 | 282-293 | 1(1, T -> |
| | | C) |
| VH3-64 | 283-294 | 0 |
| VH3-64 | 284-295 | 0 |
| 8MER | | |
| 8MER | | |
| 8MER | | |
| 8MER | | |
| 8MER | | |
| 8MER | | |
| 8MER | | |
| 8MER | | |
| 8MER | | |
| D3-22 | 01-Dec | 2(1, A -> |
| | | G); (11, T |
| | | ->G) |
| D3-22 | Feb-13 | 2(10, A -> |
| | | G); (12, |
| | | G ->C) |
| D3-22 | Mar-14 | 2(9, A -> |
| | | G); (11, |
| | | G ->C) |
| D3-22 | Apr-15 | 2(8, A -> |
| | | G); (10, |
| | | G ->C) |
| D3-22 | May-16 | 2(7, A -> |
| | | G); (9, G |
| | | ->C) |
| D3-22 | Jun-17 | 2(6, A -> |
| | | G); (8, G |
| | | ->C) |
| D3-22 | Jul-18 | 2(5, A -> |
| | | G); (7, G |
| | | ->C) |
| D3-22 | Aug-19 | 2(4, A -> |
| | | G); (6, G |
| | | ->C) |
| D3-22 | Sep-20 | 2(3, A -> |
| | | G); (5, G |
| | | ->C) |
| 8MER | | |
| 8MER | | |
| 8MER | | |
| 8MER | | |
| 8MER | | |
| 8MER | | |
| 8MER | | |
| JH3 | 01-Dec | 2(1, G -> |
| | | C); (3, G |
| | | ->A) |
| JH3 | Feb-13 | 1 (2, G -> |
| | | A) |
| JH3 | Mar-14 | 1 (1, G -> |
| | | A) |
| JH3 | Apr-15 | 0 |
| JH3 | May-16 | 0 |
| JH3 | Jun-17 | 0 |
| JH3 | Jul-18 | 0 |
| JH3 | Aug-19 | 0 |
| JH3 | Sep-20 | 0 |
| JH3 | Oct-21 | 0 |
| JH3 | Nov-22 | 0 |
| JH3 | Dec-23 | 0 |
| JH3 | 13-24 | 0 |
| JH3 | 14-25 | 0 |
| JH3 | 15-26 | 0 |
| JH3 | 16-27 | 0 |
| JH3 | 17-28 | 0 |
| JH3 | 18-29 | 1 (12, G -> |
| | | A) |
| JH3 | 19-30 | 1 (11, G -> |
| | | A) |
| JH3 | 20-31 | 1 (10, G -> |
| | | A) |
| JH3 | 21-32 | 1 (9, G -> |
| | | A) |

TABLE 9b

| | |
|---|---|
| SEQ | |
| ID: | Sequence |

| | |
|---|---|
| 61 | TGTCTACTACTGTGCGAGAGATCGTTACTATGAGACTAGTGGTT------CCAATGCTTTTGATGTCTGGGGCCAAGGAACA |
| 111 | TGTGTATTACTGTGCGAGAGA |
| 112 | GTATTACTATGATAGTAGTGGTTATTACTAC |
| 113 | GATGCTTTTGATGTCTGGGGCCAAGGGACA |
| 115 | TGTCTACTACTG |
| 116 | GTCTACTACTGT |
| 117 | TCTACTACTGTG |
| 118 | CTACTACTGTGC |
| 119 | TACTACTGTGCG |
| 120 | ACTACTGTGCGA |
| 121 | CTACTGTGCGAG |
| 122 | TACTGTGCGAGA |
| 123 | ACTGTGCGAGAGA |
| 124 | CGAGAGAT |
| 125 | GAGAGATC |
| 126 | AGAGATCG |
| 127 | GAGATCGT |
| 128 | AGATCGTT |
| 129 | GATCGTTA |
| 130 | ATCGTTAC |
| 131 | TCGTTACT |
| 132 | CGTTACTA |
| 133 | GTTACTATGAGA |
| 134 | TTACTATGAGAC |
| 135 | TACTATGAGACT |
| 136 | ACTATGAGACTA |
| 137 | CTATGAGACTAG |
| 138 | TATGAGACTAGT |
| 139 | ATGAGACTAGTG |
| 140 | TGAGACTAGTGG |
| 141 | GAGACTAGTGGT |
| 142 | TAGTGGTC |
| 143 | AGTGGTCC |
| 144 | GTGGTCCA |
| 145 | TGGTCCAA |
| 146 | GGTCCAAT |
| 147 | GTCCAATG |
| 148 | TCCAATGC |
| 149 | CCAATGCTTTTG |
| 150 | CAATGCTTTTGA |
| 151 | AATGCTTTTGAT |
| 152 | ATGCTTTTGATG |
| 153 | TGCTTTTGATGT |
| 154 | GCTTTTGATGTC |
| 155 | CTTTTGATGTCT |
| 156 | TTTTGATGTCTG |
| 157 | TTTGATGTCTGG |
| 158 | TTGATGTCTGGG |
| 159 | TGATGTCTGGGG |
| 160 | GATGTCTGGGGC |
| 161 | ATGTCTGGGGCC |
| 162 | TGTCTGGGGCCA |
| 163 | GTCTGGGGCCAA |
| 164 | TCTGGGGCCAAG |
| 165 | CTGGGGCCAAGG |
| 166 | TGGGGCCAAGGA |
| 167 | GGGGCCAAGGAA |
| 168 | GGGCCAAGGAAC |
| 169 | GGCCAAGGAACA |

G. Illustrated is the VD junction of sequence AY056842.1. All the oligos that contribute to its assembly are shown. The oligos used to construct the sequence are 11 mers (6+5) such as those used in the combinatorial SBH method however the free 5 mers were selected to completely matching VH3-64 and D3-22.

TABLE 10

| Sequence | |
|---|---|
| (oligo) | |
| name/ SEQ ID | |
| NO: | |
| AY056842.1/170 | TGTCTACTACTGTGCGAGAGATCGTTACTATGAGACTAGTGGTT |
| VH3-64/171 | TGTGTATTACTGTGCGAGAGA |
| D3-22/172 | GTATTACTATGATAGTAGTGGTT |
| 173 | |
| 174 | GTCTACTACTG |
| 175 | TCTACTACTGT |
| 176 | CTACTACTGTG |
| 177 | TACTACTGTGC |
| 178 | ACTACTGTGCG |
| 179 | CTACTGTGCGA |
| 180 | TACTGTGCGAG |
| 181 | ACTGTGCGAGA |
| 182 | CTGTGCGAGAG |
| 183 | TGTGCGAGAGA |
| 184 | AGATCGTTACT |
| 185 | GATCGTTACTA |
| 186 | ATCGTTACTAT |
| 187 | TCGTTACTATG |
| 188 | CGTTACTATGA |
| 189 | TGAGACTAGTG |
| 190 | GAGACTAGTGG |
| 191 | AGACTAGTGGT |

### Example 12

All Possible Oligonucleotides Derived from VH3-64 (Base 1 to 18) that encode for the first six amino acids.

TABLE 11

| Germline | Jan- | SEQ | E V Q L | SEQ | |
|---|---|---|---|---|---|
| VH3-64 | 18 | ID: | V E | ID: | GAGGTGCAGCTGGTGGAG |

| | | | | | |
|---|---|---|---|---|---|
| VH3-64 | 01- | 192 | E V Q L | 199 | GA[GA]GT[ATGC]CA[AG][CT]T[ATGC] |
| | Dec | | | | |
| VH3-64 | Feb- | 193 | V Q L | 200 | A[GA]GT[ATGC]CA[AG][CT]T[ATGC]G |
| | 13 | | | | |
| VH3-64 | Mar- | 194 | V Q L | 201 | [GA]GT[ATGC]CA[AG][CT]T[ATGC]GT |
| | 14 | | | | |
| VH3-64 | Apr- | 195 | V Q L | 202 | GT[ATGC]CA[AG][CT]T[ATGC]GT[ATGC] |
| | 15 | | V | | |
| VH3-64 | May- | 196 | Q L | 203 | T[ATGC]CA[AG][CT]T[ATGC]GT[ATGC]G |
| | 16 | V | | | |
| VH3-64 | Jun- | 197 | Q L | 114 | [ATGC]CA[AG][CT]T[ATGC]GT[ATGC]GA |
| | 17 | V | | | |
| VM3-64 | Jul- | 198 | Q L | 173 | CA[AG][CT]T[ATGC]GT[ATGC]GA[GA] |
| | 18 | V E | | | |

### Example 13

Preparation of Hychip Arrays for Combinatorial Sequencing by Hybridization and Sample Hybridization [Adapted from Cowie et al. (2004) Human Mutation 24:261-71]

PCR—Primers which are used to amplify a sample containing cells producing IgM antibodies with kappa light chains are described in Table 12, here in below. These primers amplify the region of the leader sequence of either the heavy of light kappa chains. The downstream primer was selected from the constant region.

TABLE 12
Primers used for RT-PCR

| | | |
|---|---|---|
| Matches (L = leader, | SEQ | |
| H = heavy, | ID | |
| K = kappa) | NO: | Sequence |

| | | |
|---|---|---|
| LH (1 + 7) | 1 | ATGGACTGSACCTGGAGVRTC |
| LH1 | 2 | ATGGACTGGATTTGGAGGATC |
| LH2 | 3 | ATGGACACACTTTGCTMCAC |
| LH3.1 | 4 | GCTGGGTTTTCCTYGTTGY |
| LH3.2 | 5 | CTGAGCTGGMTTTTYCTT |
| LH4 | 6 | CTGGTGGCRGCTCCCAGA |
| LKI | 7 | GCTCAGCTCCTGGGGCTCCTG |
| LKII | 8 | CTGGGGCTGCTAATGCTCTGG |
| LKIII | 9 | TTCCTCCTGCTACTCTGGCTC |
| LKIV | 10 | CAGACCCAGGTCTTCATTTCT |
| antisense kappa | 11 | TTTCAACTGCTCATCAGATGGCGG |
| LH1 & LH7 | 12 | CCATGGACTGGACCTGG |
| LH6 | 13 | ATGTCTGTCTCCTTCCTCAT |
| LH4 | 14 | ATGAAACACCTGTGGTTCTT |
| LH3 | 15 | CCATGGAGTTKGGGCTGAGC |
| LH5 | 16 | ATGGGGTCAACCGCCATCCT |
| LH2 | 17 | CCATGGACACACTTTGYTCCAC |
| antisense mu | 18 | AGACGAGGGGGAAAAGGGTT |

Examples of PCR conditions—PCR Conditions used are according to Bioneer AccuPower PCR PreMix Cat. No. K 2013 protocol. For the kappa primers the PCR program used is: 94° C. for 3′ followed by 34 cycles of 94° C. for 30 seconds, 55° C. for 30 seconds, 72° C. for 2 minutes and 72° C. for 7 minutes and 4° C. for hold. The heavy chain program was either 94° C. for 3 minutes followed by 9 cycles of 94° C. for 30 minutes, 46° C. for 30 seconds and 72° C. for 1.30 minutes then 25 cycles of 94° C. for 30 seconds, 48° C. for 30 seconds and 72° C. for 1.30 minutes and then 72° C. for 7 minutes and 04° C. for hold or 94° C. for 3 minutes followed by 34 cycles of 94° C. for 30 seconds, 50° C. for 30 seconds and 72° C. for 1.30 minutes and then 72° C. for 7 minutes and 04° C. for hold.

Probes—Amino modified hexamer (6 mer) probes and pentamer (5 mer) TAMRA fluorescent—labeled probes are obtained from Biosearch Technologies (Novato, CA). HyChip™ arrays (Callida Genomics, Sunnyvale, Calif.) are prepared by spotting presynthesized probes carrying a 5′-amino group followed by at least two C18 linkers. Probes usually have two to three fully degenerate positions (N) followed by 6 specific bases (B, e.g., 5′-NH₂—C18-C18-N(2-3)-BBBBBB-3′). HyChip arrays are spotted on microscope glass slides with 8 spotting areas defined by printed Teflon boundaries (Erie Scientific Soda Glass, Portsmouth, N.H.). The glass surface of a batch of up to 1200 slides is activated by forming a layer of (Si)n-NH₂ (3-propylamino silane) and amine groups are then saturated with double-sided coupling agent 1,4-phenylene diisothiocyanate. Amino-modified probes are spotted using an IAI robot (Autumation Controls Group, Campbell, CA) in 100 mM bicarbonate buffer and covalently attached to free isothiocyanate groups on the activated glass. Spot size is approximately 130 μM at 150 μM center-center spacing. After spotting, the slides are washed to remove unbound probe molecules and then blocked with protein prep (Amersham, Piscataway, N.J.). Optimized slide activation and probe spotting processes result in a very high density of bound oligonucleotides of about 100,000/μ². HyChip slides contain 8 replica arrays of a complete set of 4,096 attached 6 mer probes and hundreds of fluorescent and empty marker dots to guide image analysis. A standard universal set of 8 probe pools containing a complete set of 1,024 3′-TAMRA labeled and 5′-phosphorylated 5 mers are generated by mixing 128 specific probes per pool (Callida Genomics, Sunnyvale, Calif.). Each 5-mer probe is present in only one pool in a given set. A 3-μl to 6 μl pool aliquote used on one array contains an optimized amount between 0.1-1.0 pmol of each probe.

Template preparation—PCR products are purified using the Qiagen QIA Quick Mini columns (Qiagen, Valencia, Calif.), and eluted in 30 μl of double-distilled water. DNA concentration is obtained by electrophoresing 2 μl of purified PCR product with 2 μl of low DNA mass ladder (Invitrogen, Carlsbad, Calif.) on a 1% (w/v) agarose gel. A total 4 pmol of PCR product is used for each HyChip where possible. A total 28 μl of double stranded PCR product (1-10 μg DNA) is digested using Callida's premade frozen mix with an optimized amount of λ exonuclease (Gibco-BRL, Rockville, Md.) at 37° C. for 15-30 min, which digest the phosphorylated PCR strand leaving the unphosphorylated single strand intact. The enzyme is then inactivated at 95° C. for 5 min. Sample volume is then adjusted to 70 μl with double distilled water and denatured at 95° C. for 5 minutes followed by 5 minutes on ice.

Hybridization—Single-stranded DNA target (67 μl) is added to a frozen aliquot of Callida's premade and pretested ligation mix (60 μl), which contains ligation buffer (New England Biolabs, Beverly, Mass.) and an optimized amount of T4 DNA ligase (New England Biolabs, Beverly, Mass.). The absolute amount of T4 which is used varies from batch to batch. A total of 14 μl of DNA-ligase mix is then dispensed into each of the 8 labeled 5-mer probe pools prealiquotted in a strip of 8 small tubes which can be obtained from Callida Genomics supra. 8 probe pools containing target DNA and ligase are mixed thoroughly and 18 μl is loaded by pipette onto the HyChip cartridge, where it is drawn by capillary forces into the selected hybridization chamber. The cartridge has a metal holder into which a glass slide and a plastic cover with 8 loading holes are inserted. The flat plastic cover has 0.5-mm wide grooves matching Teflon strips on the glass slide, and forms on the glass slide isolated eight capillary (50 μm high) hybridization chambers. The combined forces of air-filled grooves and hydrophilic Teflon prevents liquid spill between the chambers. Hybridization and ligation occurs in a humid box at room temperature for 60 min. The slides are then hot-washed to remove nonligated labeled probe in Callida's pre-made detergent (containing washing buffer) for 10 min in an orbital shaker at 80° C., and then rinsed four times in double distilled water and spin-dried. Slides are then scanned at 20 μm resolution using a standard array reader.

Analysis—Integrated software developed by Callida Genomics was then used to process hybridization results (further described by Cowie 2004, supra) and assign raw values for each probe (i.e., subtraction of background, replicate normalization, calculation of average or median for the probes in case of replicates). Sequences are compiled as described in Example 14 below.

### Example 14

Assembly Algorithm

A. Discover probes—find the set of high scoring probes (featured by X fold signal over an average probe or over the background signal).

B. Discover the germline segments—find high scoring germline segments effected by either of the following:

1. For the set of probe of (A) identifying the most likely germline segments from which the probe sequence is derived. For example, for an amplified heavy chain, identifying gene segments of VDJ by summing all scores of the individual none mutated probes which match the gene segments.

2. Scoring all relevant germline segments by summing all scores of the individual none mutated probes which match them, negating prior probe selection.

3. Use only discriminative probes which bind uniquely to and in a germline segment.

4. Weighing a probe by redundancy thereof. Thus redundant probes are assigned with a poor weight and a score thereof is reduced. For example, for an 11-mer probe capable of hybridizing to 10 different VH genes dividing its score by 10. This allows full coverage of the sequence while generating no gaps and allowing accurate discrimination between the germline genes.

C. Fine tune germiline segments—Given a certain high scoring germline segment, identifying overlapping mutated probes featured by higher score than the non-mutated probes. Upon such an event, replacing the high scoring probes with the non-mutated probes.

D. Identifying junction sequences—Identify high scoring probes which contain a 5′ or 3′ end overlap with the high scoring fine tuned germline segments. If such probes are available which link VJ or VDJ of a certain chain, a linear and complete sequence may be generated. If not, additional probes are sought.

E. Final target sequence and score—Once A-D is completed, all relevant probes are aligned in the correct order. This information allows determining the sequence and provide it with a score.

F. Identifying the sequence type ratio—Given the different scores of the assembled sequences, it is possible to calculate their relative ratio or percentile distribution.

While the present invention has been particularly described, persons skilled in the art will appreciate that many variations and modifications can be made. Therefore, the invention is not to be construed as restricted to the particularly described embodiments, rather the scope, spirit and concept of the invention will be more readily understood by reference to the claims which follow.

While the present invention has been particularly described, persons skilled in the art will appreciate that many variations and modifications can be made. Therefore, the invention is not to be construed as restricted to the particularly described embodiments, rather the scope, spirit and concept of the invention will be more readily understood by reference to the claims which follow.

1. A method of sequencing a population of polynucleotides encoding antibodies or T-cell receptors, the method compromising:
(a) contacting a plurality of oligonucleotides of known sequences, at least a portion of said plurality of oligonucleotides having a partial sequence similarity, with the population of polynucleotides under conditions allowing a formation of hybridization duplexes between at least a portion of said plurality of oligonucleotides and said population of polynucleotides;
(b) quantitavely detecting oligonucleotides involved in said formation of said hybridization duplexes; and
(c) compiling a set of sequences of the population of polynucleotides by:
(i) identifying oligonucleotides involved in said formation of said hybridization duplexes in a quantum above a predetermined threshold, so as to define a population of positively hybridizing oligonucleotides;
(ii) identifying germline sequences of germline segments in at least a portion of said positively hybridizing oligonucleotides or in at least a portion of said oligonucleotides involved in said formation of said hybridization complexes;
(iii) (iii) identifying positively hybridizing oligonucleotides overlapping with said germline sequences, thereby identifying germline junction sequences; and
(iv) assembling sequence information obtained from steps (ii) and (iii), thereby compiling said set of sequences of the population of polynucleotides;
thereby sequencing the population of polynucleotides encoding the antibodies or T-cell receptors.

2. The method of claim 1, wherein step (ii) further comprises identifying oligonucleotides of non-redundant germline sequences in said germline segment.

3. The method of claim 1, wherein each oligonucleotide of said at least a portion of said plurality of oligonucleotides is selected to hybridize with a known germline segment and an unknown sequence.

4. The method of claim 1, wherein said plurality of oligonucleotides are collectively selected to hybridize with all germline segments of the population of polynucleotides.

5. The method of claim 1, wherein said plurality of oligonucleotides is selected to non-redundantly hybridize with said germline segments of the population of polynucleotides.

6-9. (canceled)

10. The method of claim 1, wherein the population of polynucleotides comprise RNA molecules.

11. The method of claim 1, wherein the population of polynucleotides comprise DNA molecules.

12. The method of claim 1, the method further comprising, prior to step (a), amplifying selected segments of said polynucleotides encoding the antibodies or the T-cell receptors, said selected segments encoding variable regions of the antibodies or the T cell receptors.

13. The method of claim 12, wherein said variable regions comprise variable regions of heavy chains of antibodies.

14. The method of claim 12, wherein said variable regions comprise variable regions of light chains of antibodies.

15-19. (canceled)

20. A method of quantifying an expression of a population of polynucleotides encoding antibodies or T-cell receptors, the method comprising:
(a) contacting a plurality of oligonucleotides of known sequences, at least a portion of said plurality of oligonucleotides having a partial sequence similarity, with the population of polynucleotides under conditions allowing a formation of hybridization duplexes between at least a portion of said plurality of oligonucleotides and said population of polynucleotides;
(b) quantitavely detecting oligonucleotides involved in said formation of said hybridization duplexes; and
(c) compiling a set of sequences of the population of polynucleotides by:
(i) identifying oligonucleotides involved in said formation of said hybridization duplexes in a quantum above a predetermined threshold, so as to define a population of positively hybridizing oligonucleotides;
(ii) identifying germline sequences of germline segments in at least a portion of said positively hybridizing oligonucleotides or in at least a portion of said oligonucleotides involved in said formation of said hybridization complexes;
(iii) identifying positively hybridizing oligonucleotides overlapping with said germline sequences, thereby identifying germline junction sequences;
(iv) assembling sequence information obtained from steps (ii) and (iii), thereby compiling said set of sequences of the population of polynucleotides, and
(d) determining a level of each set of said compiled set of sequences of step (iv) in said population of polynucleotides, thereby quantifying the expression of the population of polynucleotides encoding the antibodies or T-cell receptors.

21. The method of claim 20, wherein step (ii) further comprises identifying oligonucleotides of non-redundant germline sequences in said germline segment.

22. The method of claim 20, wherein each oligonucleotide of said at least a portion of said plurality of oligonucleotides is selected to hybridize with a known germline segment and an unknown sequence.

23. The method of claim 20, wherein said plurality of oligonucleotides are collectively selected to hybridize with all germline segments of the population of polynucleotides.

24-28. (canceled)

29. The method of claim 20, wherein the population of polynucleotides comprise RNA molecules.

30. The method of claim 20, wherein the population of polynucleotides comprise DNA molecules.

31. The method of claim 20, the method further comprising, prior to step (a), amplifying selected segments of said polynucleotides encoding the antibodies or the T-cell receptors, said selected segments encoding variable regions of the antibodies or the T cell receptors.

32. The method of claim 31, wherein said variable regions comprise variable regions of heavy chains of antibodies.

33. The method of claim 31, wherein said variable regions comprise variable regions of light chains of antibodies.

34-38. (canceled)

39. An oligonucleotide library for sequencing by hybridization of polynucleotides encoding variable regions of antibodies or T cell receptors, the library consisting essentially of:
(i) a set of overlapping oligonucleotides collectively selected to hybridize with all germline segments encoding the variable regions of the antibodies or T cell receptors under conditions allowing formation of hybridization duplexes between said set of overlapping oligonucleotides and said polynucleotides; and
(ii) a variant set of oligonucleotides of said overlapping oligonucleotides which comprises (G,C,T,A) base variation in at least one position of said overlapping oligonucleotides;
wherein oligonucleotides of said sets of overlapping oligonucleotides and said variant set of oligonucleotides are N bases in length and said overlapping is of at least N−(N−1) and whereas N is an integer equal or greater than 5.

40-46. (canceled)


SEQUENCE LISTING (203 sequences)

SEQ ID NO: 1 - DNA, 21 bp, Artificial sequence
  Single strand DNA oligonucleotide
atggactgsacctggagvrtc

SEQ ID NO: 2 - DNA, 21 bp, Artificial sequence
  Single strand DNA oligonucleotide
atggactggatttggaggatc

SEQ ID NO: 3 - DNA, 20 bp, Artificial sequence
  Single strand DNA oligonucleotide
atggacacactttgctmcac

SEQ ID NO: 4 - DNA, 19 bp, Artificial sequence
  Single strand DNA oligonucleotide
gctgggttttcctygttgy

SEQ ID NO: 5 - DNA, 18 bp, Artificial sequence
  Single strand DNA oligonucleotide
ctgagctggmttttyctt

SEQ ID NO: 6 - DNA, 18 bp, Artificial sequence
  Single strand DNA oligonucleotide
ctggtggcrgctcccaga

SEQ ID NO: 7 - DNA, 21 bp, Artificial sequence
  Single strand DNA oligonucleotide
gctcagctcctggggctcctg

SEQ ID NO: 8 - DNA, 21 bp, Artificial sequence
  Single strand DNA oligonucleotide
ctggggctgctaatgctctgg

SEQ ID NO: 9 - DNA, 21 bp, Artificial sequence
  Single strand DNA oligonucleotide
ttcctcctgctactctggctc

SEQ ID NO: 10 - DNA, 21 bp, Artificial sequence
  Single strand DNA oligonucleotide
cagacccaggtcttcatttct

SEQ ID NO: 11 - DNA, 24 bp, Artificial sequence
  Single strand DNA oligonucleotide
tttcaactgctcatcagatggcgg

SEQ ID NO: 12 - DNA, 17 bp, Artificial sequence
  Single strand DNA oligonucleotide
ccatggactggacctgg

SEQ ID NO: 13 - DNA, 20 bp, Artificial sequence
  Single strand DNA oligonucleotide
atgtctgtctccttcctcat

SEQ ID NO: 14 - DNA, 20 bp, Artificial sequence
  Single strand DNA oligonucleotide
atgaaacacctgtggttctt

SEQ ID NO: 15 - DNA, 20 bp, Artificial sequence
  Single strand DNA oligonucleotide
ccatggagttkgggctgagc

SEQ ID NO: 16 - DNA, 20 bp, Artificial sequence
  Single strand DNA oligonucleotide
atggggtcaaccgccatcct

SEQ ID NO: 17 - DNA, 22 bp, Artificial sequence
  Single strand DNA oligonucleotide
ccatggacacactttgytccac

SEQ ID NO: 18 - DNA, 20 bp, Artificial sequence
  Single strand DNA oligonucleotide
agacgagggggaaaagggtt

SEQ ID NO: 19 - DNA, 12 bp, Artificial sequence
  Single strand DNA oligonucleotide
caggttcagctg

SEQ ID NO: 20 - DNA, 12 bp, Artificial sequence
  Single strand DNA oligonucleotide
gaggttcagctg

SEQ ID NO: 21 - DNA, 12 bp, Artificial sequence
  Single strand DNA oligonucleotide
aaggttcagctg

SEQ ID NO: 22 - DNA, 12 bp, Artificial sequence
  Single strand DNA oligonucleotide
taggttcagctg

SEQ ID NO: 23 - DNA, 12 bp, Artificial sequence
  Single strand DNA oligonucleotide
ccggttcagctg

SEQ ID NO: 24 - DNA, 12 bp, Artificial sequence
  Single strand DNA oligonucleotide
cgggttcagctg

SEQ ID NO: 25 - DNA, 12 bp, Artificial sequence
  Single strand DNA oligonucleotide
ctggttcagctg

SEQ ID NO: 26 - DNA, 12 bp, Artificial sequence
  Single strand DNA oligonucleotide
cacgttcagctg

SEQ ID NO: 27 - DNA, 12 bp, Artificial sequence
  Single strand DNA oligonucleotide
caagttcagctg

SEQ ID NO: 28 - DNA, 12 bp, Artificial sequence
  Single strand DNA oligonucleotide
catgttcagctg

SEQ ID NO: 29 - DNA, 12 bp, Artificial sequence
  Single strand DNA oligonucleotide
cagcttcagctg

SEQ ID NO: 30 - DNA, 12 bp, Artificial sequence
  Single strand DNA oligonucleotide
cagattcagctg

SEQ ID NO: 31 - DNA, 12 bp, Artificial sequence
  Single strand DNA oligonucleotide
cagtttcagctg

SEQ ID NO: 32 - DNA, 12 bp, Artificial sequence
  Single strand DNA oligonucleotide
caggatcagctg

SEQ ID NO: 33 - DNA, 12 bp, Artificial sequence
  Single strand DNA oligonucleotide
caggctcagctg

SEQ ID NO: 34 - DNA, 12 bp, Artificial sequence
  Single strand DNA oligonucleotide
cagggtcagctg

SEQ ID NO: 35 - DNA, 12 bp, Artificial sequence
  Single strand DNA oligonucleotide
caggtacagctg

SEQ ID NO: 36 - DNA, 12 bp, Artificial sequence
  Single strand DNA oligonucleotide
caggtccagctg

SEQ ID NO: 37 - DNA, 12 bp, Artificial sequence
  Single strand DNA oligonucleotide
caggtgcagctg

SEQ ID NO: 38 - DNA, 12 bp, Artificial sequence
  Single strand DNA oligonucleotide
caggttaagctg

SEQ ID NO: 39 - DNA, 12 bp, Artificial sequence
  Single strand DNA oligonucleotide
caggtttagctg

SEQ ID NO: 40 - DNA, 12 bp, Artificial sequence
  Single strand DNA oligonucleotide
caggttgagctg

SEQ ID NO: 41 - DNA, 12 bp, Artificial sequence
  Single strand DNA oligonucleotide
caggttctgctg

SEQ ID NO: 42 - DNA, 12 bp, Artificial sequence
  Single strand DNA oligonucleotide
caggttccgctg

SEQ ID NO: 43 - DNA, 12 bp, Artificial sequence
  Single strand DNA oligonucleotide
caggttcggctg

SEQ ID NO: 44 - DNA, 12 bp, Artificial sequence
  Single strand DNA oligonucleotide
caggttcacctg

SEQ ID NO: 45 - DNA, 12 bp, Artificial sequence
  Single strand DNA oligonucleotide
caggttcaactg

SEQ ID NO: 46 - DNA, 12 bp, Artificial sequence
  Single strand DNA oligonucleotide
caggttcatctg

SEQ ID NO: 47 - DNA, 12 bp, Artificial sequence
  Single strand DNA oligonucleotide
caggttcaggtg

SEQ ID NO: 48 - DNA, 12 bp, Artificial sequence
  Single strand DNA oligonucleotide
caggttcagatg

SEQ ID NO: 49 - DNA, 12 bp, Artificial sequence
  Single strand DNA oligonucleotide
caggttcagttg

SEQ ID NO: 50 - DNA, 12 bp, Artificial sequence
  Single strand DNA oligonucleotide
caggttcagcag

SEQ ID NO: 51 - DNA, 12 bp, Artificial sequence
  Single strand DNA oligonucleotide
caggttcagccg

SEQ ID NO: 52 - DNA, 12 bp, Artificial sequence
  Single strand DNA oligonucleotide
caggttcagcgg

SEQ ID NO: 53 - DNA, 12 bp, Artificial sequence
  Single strand DNA oligonucleotide
caggttcagcta

SEQ ID NO: 54 - DNA, 12 bp, Artificial sequence
  Single strand DNA oligonucleotide
caggttcagctc

SEQ ID NO: 55 - DNA, 12 bp, Artificial sequence
  Single strand DNA oligonucleotide
caggttcagctt

SEQ ID NO: 56 - DNA, 31 bp, Artificial sequence
  Single strand DNA oligonucleotide
ctccgtcagcagtggtggttactactggagc

SEQ ID NO: 57 - DNA, 31 bp, Artificial sequence
  Single strand DNA oligonucleotide
ctccatcagcagtagtagttactactggggc

SEQ ID NO: 58 - DNA, 31 bp, Artificial sequence
  Single strand DNA oligonucleotide
ctccgtcagcagtagtagttactactggagc

SEQ ID NO: 59 - DNA, 82 bp, Artificial sequence
  Single strand DNA oligonucleotide
  misc_feature (45)..(50): n is a, c, g, or t
tgtctactactgtgcgagagatcgttactatgagactagtggttnnnnnnccaatgcttttgatgtctggggccaaggaaca

SEQ ID NO: 60 - DNA, 11 bp, Artificial sequence
  Single strand DNA oligonucleotide
tgtgcgagaga

SEQ ID NO: 61 - DNA, 17 bp, Artificial sequence
  Single strand DNA oligonucleotide
ggtacaactggaacgac

SEQ ID NO: 62 - DNA, 59 bp, Artificial sequence
  Single strand DNA oligonucleotide
aggtgcagctggtgcagtctgggggaggcctagtccagccgggggggtccctgagactc

SEQ ID NO: 63 - DNA, 12 bp, Artificial sequence
  Single strand DNA oligonucleotide
aggtgcagctgg

SEQ ID NO: 64 - DNA, 12 bp, Artificial sequence
  Single strand DNA oligonucleotide
ggtgcagctggt

SEQ ID NO: 65 - DNA, 12 bp, Artificial sequence
  Single strand DNA oligonucleotide
gtgcagctggtg

SEQ ID NO: 66 - DNA, 12 bp, Artificial sequence
  Single strand DNA oligonucleotide
tgcagctggtgc

SEQ ID NO: 67 - DNA, 12 bp, Artificial sequence
  Single strand DNA oligonucleotide
gcagctggtgca

SEQ ID NO: 68 - DNA, 12 bp, Artificial sequence
  Single strand DNA oligonucleotide
cagctggtgcag

SEQ ID NO: 69 - DNA, 12 bp, Artificial sequence
  Single strand DNA oligonucleotide
agctggtgcagt

SEQ ID NO: 70 - DNA, 12 bp, Artificial sequence
  Single strand DNA oligonucleotide
gctggtgcagtc

SEQ ID NO: 71 - DNA, 12 bp, Artificial sequence
  Single strand DNA oligonucleotide
ctggtgcagtct

SEQ ID NO: 72 - DNA, 12 bp, Artificial sequence
  Single strand DNA oligonucleotide
tggtgcagtctg

SEQ ID NO: 73 - DNA, 12 bp, Artificial sequence
  Single strand DNA oligonucleotide
ggtgcagtctgg

SEQ ID NO: 74 - DNA, 12 bp, Artificial sequence
  Single strand DNA oligonucleotide
gtgcagtctggg

SEQ ID NO: 75 - DNA, 12 bp, Artificial sequence
  Single strand DNA oligonucleotide
tgcagtctgggg

SEQ ID NO: 76 - DNA, 12 bp, Artificial sequence
  Single strand DNA oligonucleotide
gcagtctggggg

SEQ ID NO: 77 - DNA, 12 bp, Artificial sequence
  Single strand DNA oligonucleotide
cagtctggggga

SEQ ID NO: 78 - DNA, 12 bp, Artificial sequence
  Single strand DNA oligonucleotide
agtctgggggag

SEQ ID NO: 79 - DNA, 12 bp, Artificial sequence
  Single strand DNA oligonucleotide
gtctgggggagg

SEQ ID NO: 80 - DNA, 12 bp, Artificial sequence
  Single strand DNA oligonucleotide
tctgggggaggc

SEQ ID NO: 81 - DNA, 12 bp, Artificial sequence
  Single strand DNA oligonucleotide
ctgggggaggcc

SEQ ID NO: 82 - DNA, 12 bp, Artificial sequence
  Single strand DNA oligonucleotide
tgggggaggcct

SEQ ID NO: 83 - DNA, 12 bp, Artificial sequence
  Single strand DNA oligonucleotide
gggggaggccta

SEQ ID NO: 84 - DNA, 12 bp, Artificial sequence
  Single strand DNA oligonucleotide
ggggaggcctag

SEQ ID NO: 85 - DNA, 12 bp, Artificial sequence
  Single strand DNA oligonucleotide
gggaggcctagt

SEQ ID NO: 86 - DNA, 12 bp, Artificial sequence
  Single strand DNA oligonucleotide
ggaggcctagtc

SEQ ID NO: 87 - DNA, 12 bp, Artificial sequence
  Single strand DNA oligonucleotide
gaggcctagtcc

SEQ ID NO: 88 - DNA, 12 bp, Artificial sequence
  Single strand DNA oligonucleotide
aggcctagtcca

SEQ ID NO: 89 - DNA, 12 bp, Artificial sequence
  Single strand DNA oligonucleotide
ggcctagtccag

SEQ ID NO: 90 - DNA, 12 bp, Artificial sequence
  Single strand DNA oligonucleotide
gcctagtccagc

SEQ ID NO: 91 - DNA, 12 bp, Artificial sequence
  Single strand DNA oligonucleotide
cctagtccagcc

SEQ ID NO: 92 - DNA, 12 bp, Artificial sequence
  Single strand DNA oligonucleotide
ctagtccagccg

SEQ ID NO: 93 - DNA, 12 bp, Artificial sequence
  Single strand DNA oligonucleotide
tagtccagccgg

SEQ ID NO: 94 - DNA, 12 bp, Artificial sequence
  Single strand DNA oligonucleotide
agtccagccggg

SEQ ID NO: 95 - DNA, 12 bp, Artificial sequence
  Single strand DNA oligonucleotide
gtccagccgggg

SEQ ID NO: 96 - DNA, 12 bp, Artificial sequence
  Single strand DNA oligonucleotide
tccagccggggg

SEQ ID NO: 97 - DNA, 12 bp, Artificial sequence
  Single strand DNA oligonucleotide
ccagccgggggg

SEQ ID NO: 98 - DNA, 12 bp, Artificial sequence
  Single strand DNA oligonucleotide
cagccggggggg

SEQ ID NO: 99 - DNA, 12 bp, Artificial sequence
  Single strand DNA oligonucleotide
agccgggggggt

SEQ ID NO: 100 - DNA, 12 bp, Artificial sequence
  Single strand DNA oligonucleotide
gccgggggggtc

SEQ ID NO: 101 - DNA, 12 bp, Artificial sequence
  Single strand DNA oligonucleotide
ccgggggggtcc

SEQ ID NO: 102 - DNA, 12 bp, Artificial sequence
  Single strand DNA oligonucleotide
cgggggggtccc

SEQ ID NO: 103 - DNA, 12 bp, Artificial sequence
  Single strand DNA oligonucleotide
gggggggtccct

SEQ ID NO: 104 - DNA, 12 bp, Artificial sequence
  Single strand DNA oligonucleotide
ggggggtccctg

SEQ ID NO: 105 - DNA, 12 bp, Artificial sequence
  Single strand DNA oligonucleotide
gggggtccctga

SEQ ID NO: 106 - DNA, 12 bp, Artificial sequence
  Single strand DNA oligonucleotide
ggggtccctgag

SEQ ID NO: 107 - DNA, 12 bp, Artificial sequence
  Single strand DNA oligonucleotide
gggtccctgaga

SEQ ID NO: 108 - DNA, 12 bp, Artificial sequence
  Single strand DNA oligonucleotide
ggtccctgagac

SEQ ID NO: 109 - DNA, 12 bp, Artificial sequence
  Single strand DNA oligonucleotide
gtccctgagact

SEQ ID NO: 110 - DNA, 12 bp, Artificial sequence
  Single strand DNA oligonucleotide
tccctgagactc

SEQ ID NO: 111 - DNA, 21 bp, Artificial sequence
  Single strand DNA oligonucleotide
tgtgtattactgtgcgagaga

SEQ ID NO: 112 - DNA, 31 bp, Artificial sequence
  Single strand DNA oligonucleotide
gtattactatgatagtagtggttattactac

SEQ ID NO: 113 - DNA, 30 bp, Artificial sequence
  Single strand DNA oligonucleotide
gatgcttttgatgtctggggccaagggaca

SEQ ID NO: 114 - DNA, 12 bp, Artificial sequence
  Single strand DNA oligonucleotide
  misc_feature (1)..(1): n is a, c, g, or t
  misc_feature (7)..(7): n is a, c, g, or t
  misc_feature (10)..(10): n is a, c, g, or t
ncarytngtnga

SEQ ID NO: 115 - DNA, 12 bp, Artificial sequence
  Single strand DNA oligonucleotide
tgtctactactg

SEQ ID NO: 116 - DNA, 12 bp, Artificial sequence
  Single strand DNA oligonucleotide
gtctactactgt

SEQ ID NO: 117 - DNA, 12 bp, Artificial sequence
  Single strand DNA oligonucleotide
tctactactgtg

SEQ ID NO: 118 - DNA, 12 bp, Artificial sequence
  Single strand DNA oligonucleotide
ctactactgtgc

SEQ ID NO: 119 - DNA, 12 bp, Artificial sequence
  Single strand DNA oligonucleotide
tactactgtgcg

SEQ ID NO: 120 - DNA, 12 bp, Artificial sequence
  Single strand DNA oligonucleotide
actactgtgcga

SEQ ID NO: 121 - DNA, 12 bp, Artificial sequence
  Single strand DNA oligonucleotide
ctactgtgcgag

SEQ ID NO: 122 - DNA, 12 bp, Artificial sequence
  Single strand DNA oligonucleotide
tactgtgcgaga

SEQ ID NO: 123 - DNA, 13 bp, Artificial sequence
  Single strand DNA oligonucleotide
actgtgcgagaga

SEQ ID NO: 124 - DNA, 8 bp, Artificial sequence
  Single strand DNA oligonucleotide
cgagagat

SEQ ID NO: 125 - DNA, 8 bp, Artificial sequence
  Single strand DNA oligonucleotide
gagagatc

SEQ ID NO: 126 - DNA, 8 bp, Artificial sequence
  Single strand DNA oligonucleotide
agagatcg

SEQ ID NO: 127 - DNA, 8 bp, Artificial sequence
  Single strand DNA oligonucleotide
gagatcgt

SEQ ID NO: 128 - DNA, 8 bp, Artificial sequence
  Single strand DNA oligonucleotide
agatcgtt

SEQ ID NO: 129 - DNA, 8 bp, Artificial sequence
  Single strand DNA oligonucleotide
gatcgtta

SEQ ID NO: 130 - DNA, 8 bp, Artificial sequence
  Single strand DNA oligonucleotide
atcgttac

SEQ ID NO: 131 - DNA, 8 bp, Artificial sequence
  Single strand DNA oligonucleotide
tcgttact

SEQ ID NO: 132 - DNA, 8 bp, Artificial sequence
  Single strand DNA oligonucleotide
cgttacta

SEQ ID NO: 133 - DNA, 12 bp, Artificial sequence
  Single strand DNA oligonucleotide
gttactatgaga

SEQ ID NO: 134 - DNA, 12 bp, Artificial sequence
  Single strand DNA oligonucleotide
ttactatgagac

SEQ ID NO: 135 - DNA, 12 bp, Artificial sequence
  Single strand DNA oligonucleotide
tactatgagact

SEQ ID NO: 136 - DNA, 12 bp, Artificial sequence
  Single strand DNA oligonucleotide
actatgagacta

SEQ ID NO: 137 - DNA, 12 bp, Artificial sequence
  Single strand DNA oligonucleotide
ctatgagactag

SEQ ID NO: 138 - DNA, 12 bp, Artificial sequence
  Single strand DNA oligonucleotide
tatgagactagt

SEQ ID NO: 139 - DNA, 12 bp, Artificial sequence
  Single strand DNA oligonucleotide
atgagactagtg

SEQ ID NO: 140 - DNA, 12 bp, Artificial sequence
  Single strand DNA oligonucleotide
tgagactagtgg

SEQ ID NO: 141 - DNA, 12 bp, Artificial sequence
  Single strand DNA oligonucleotide
gagactagtggt

SEQ ID NO: 142 - DNA, 8 bp, Artificial sequence
  Single strand DNA oligonucleotide
tagtggtc

SEQ ID NO: 143 - DNA, 8 bp, Artificial sequence
  Single strand DNA oligonucleotide
agtggtcc

SEQ ID NO: 144 - DNA, 8 bp, Artificial sequence
  Single strand DNA oligonucleotide
gtggtcca

SEQ ID NO: 145 - DNA, 8 bp, Artificial sequence
  Single strand DNA oligonucleotide
tggtccaa

SEQ ID NO: 146 - DNA, 8 bp, Artificial sequence
  Single strand DNA oligonucleotide
ggtccaat

SEQ ID NO: 147 - DNA, 8 bp, Artificial sequence
  Single strand DNA oligonucleotide
gtccaatg

SEQ ID NO: 148 - DNA, 8 bp, Artificial sequence
  Single strand DNA oligonucleotide
tccaatgc

SEQ ID NO: 149 - DNA, 12 bp, Artificial sequence
  Single strand DNA oligonucleotide
ccaatgcttttg

SEQ ID NO: 150 - DNA, 12 bp, Artificial sequence
  Single strand DNA oligonucleotide
caatgcttttga

SEQ ID NO: 151 - DNA, 12 bp, Artificial sequence
  Single strand DNA oligonucleotide
aatgcttttgat

SEQ ID NO: 152 - DNA, 12 bp, Artificial sequence
  Single strand DNA oligonucleotide
atgcttttgatg

SEQ ID NO: 153 - DNA, 12 bp, Artificial sequence
  Single strand DNA oligonucleotide
tgcttttgatgt

SEQ ID NO: 154 - DNA, 12 bp, Artificial sequence
  Single strand DNA oligonucleotide
gcttttgatgtc

SEQ ID NO: 155 - DNA, 12 bp, Artificial sequence
  Single strand DNA oligonucleotide
cttttgatgtct

SEQ ID NO: 156 - DNA, 12 bp, Artificial sequence
  Single strand DNA oligonucleotide
ttttgatgtctg

SEQ ID NO: 157 - DNA, 12 bp, Artificial sequence
  Single strand DNA oligonucleotide
tttgatgtctgg

SEQ ID NO: 158 - DNA, 12 bp, Artificial sequence
  Single strand DNA oligonucleotide
ttgatgtctggg

SEQ ID NO: 159 - DNA, 12 bp, Artificial sequence
  Single strand DNA oligonucleotide
tgatgtctgggg

SEQ ID NO: 160 - DNA, 12 bp, Artificial sequence
  Single strand DNA oligonucleotide
gatgtctggggc

SEQ ID NO: 161 - DNA, 12 bp, Artificial sequence
  Single strand DNA oligonucleotide
atgtctggggcc

SEQ ID NO: 162 - DNA, 12 bp, Artificial sequence
  Single strand DNA oligonucleotide
tgtctggggcca

SEQ ID NO: 163 - DNA, 12 bp, Artificial sequence
  Single strand DNA oligonucleotide
gtctggggccaa

SEQ ID NO: 164 - DNA, 12 bp, Artificial sequence
  Single strand DNA oligonucleotide
tctggggccaag

SEQ ID NO: 165 - DNA, 12 bp, Artificial sequence
  Single strand DNA oligonucleotide
ctggggccaagg

SEQ ID NO: 166 - DNA, 12 bp, Artificial sequence
  Single strand DNA oligonucleotide
tggggccaagga

SEQ ID NO: 167 - DNA, 12 bp, Artificial sequence
  Single strand DNA oligonucleotide
ggggccaaggaa

SEQ ID NO: 168 - DNA, 12 bp, Artificial sequence
  Single strand DNA oligonucleotide
gggccaaggaac

SEQ ID NO: 169 - DNA, 12 bp, Artificial sequence
  Single strand DNA oligonucleotide
ggccaaggaaca

SEQ ID NO: 170 - DNA, 44 bp, Artificial sequence
  Single strand DNA oligonucleotide
tgtctactactgtgcgagagatcgttactatgagactagtggtt

SEQ ID NO: 171 - DNA, 21 bp, Artificial sequence
  Single strand DNA oligonucleotide
tgtgtattactgtgcgagaga

SEQ ID NO: 172 - DNA, 23 bp, Artificial sequence
  Single strand DNA oligonucleotide
gtattactatgatagtagtggtt

SEQ ID NO: 173 - DNA, 12 bp, Artificial sequence
  Single strand DNA oligonucleotide
  misc_feature (6)..(6): n is a, c, g, or t
  misc_feature (9)..(9): n is a, c, g, or t
carytngtngar

SEQ ID NO: 174 - DNA, 11 bp, Artificial sequence
  Single strand DNA oligonucleotide
gtctactactg

SEQ ID NO: 175 - DNA, 11 bp, Artificial sequence
  Single strand DNA oligonucleotide
tctactactgt

SEQ ID NO: 176 - DNA, 11 bp, Artificial sequence
  Single strand DNA oligonucleotide
ctactactgtg

SEQ ID NO: 177 - DNA, 11 bp, Artificial sequence
  Single strand DNA oligonucleotide
tactactgtgc

SEQ ID NO: 178 - DNA, 11 bp, Artificial sequence
  Single strand DNA oligonucleotide
actactgtgcg

SEQ ID NO: 179 - DNA, 11 bp, Artificial sequence
  Single strand DNA oligonucleotide
ctactgtgcga

SEQ ID NO: 180 - DNA, 11 bp, Artificial sequence
  Single strand DNA oligonucleotide
tactgtgcgag

SEQ ID NO: 181 - DNA, 11 bp, Artificial sequence
  Single strand DNA oligonucleotide
actgtgcgaga

SEQ ID NO: 182 - DNA, 11 bp, Artificial sequence
  Single strand DNA oligonucleotide
ctgtgcgagag

SEQ ID NO: 183 - DNA, 11 bp, Artificial sequence
  Single strand DNA oligonucleotide
tgtgcgagaga

SEQ ID NO: 184 - DNA, 11 bp, Artificial sequence
  Single strand DNA oligonucleotide
agatcgttact

SEQ ID NO: 185 - DNA, 11 bp, Artificial sequence
  Single strand DNA oligonucleotide
gatcgttacta

SEQ ID NO: 186 - DNA, 11 bp, Artificial sequence
  Single strand DNA oligonucleotide
atcgttactat

SEQ ID NO: 187 - DNA, 11 bp, Artificial sequence
  Single strand DNA oligonucleotide
tcgttactatg

SEQ ID NO: 188 - DNA, 11 bp, Artificial sequence
  Single strand DNA oligonucleotide
cgttactatga

SEQ ID NO: 189 - DNA, 11 bp, Artificial sequence
  Single strand DNA oligonucleotide
tgagactagtg

SEQ ID NO: 190 - DNA, 11 bp, Artificial sequence
  Single strand DNA oligonucleotide
gagactagtgg

SEQ ID NO: 191 - DNA, 11 bp, Artificial sequence
  Single strand DNA oligonucleotide
agactagtggt

SEQ ID NO: 192 - protein, 4 aa, Artificial sequence
  Peptide
EVQL

SEQ ID NO: 193 - protein, 3 aa, Artificial sequence
  Peptide
VQL

SEQ ID NO: 194 - protein, 3 aa, Artificial sequence
  Peptide
VQL

SEQ ID NO: 195 - protein, 4 aa, Artificial sequence
  Peptide
VQLV

SEQ ID NO: 196 - protein, 3 aa, Artificial sequence
  Peptide
QLV

SEQ ID NO: 197 - protein, 3 aa, Artificial sequence
  Peptide
QLV

SEQ ID NO: 198 - protein, 4 aa, Artificial sequence
  Peptide
QLVE

SEQ ID NO: 199 - DNA, 12 bp, Artificial sequence
  Single strand DNA oligonucleotide
  misc_feature (6)..(6): n is a, c, g, or t
  misc_feature (12)..(12): n is a, c, g, or t
gargtncarytn

SEQ ID NO: 200 - DNA, 12 bp, Artificial sequence
  Single strand DNA oligonucleotide
  misc_feature (5)..(5): n is a, c, g, or t
  misc_feature (11)..(11): n is a, c, g, or t
argtncarytng

SEQ ID NO: 201 - DNA, 12 bp, Artificial sequence
  Single strand DNA oligonucleotide
  misc_feature (4)..(4): n is a, c, g, or t
  misc_feature (10)..(10): n is a, c, g, or t
rgtncarytngt

SEQ ID NO: 202 - DNA, 12 bp, Artificial sequence
  Single strand DNA oligonucleotide
  misc_feature (3)..(3): n is a, c, g, or t
  misc_feature (9)..(9): n is a, c, g, or t
  misc_feature (12)..(12): n is a, c, g, or t
gtncarytngtn

SEQ ID NO: 203 - DNA, 12 bp, Artificial sequence
  Single strand DNA oligonucleotide
  misc_feature (2)..(2): n is a, c, g, or t
  misc_feature (8)..(8): n is a, c, g, or t
  misc_feature (11)..(11): n is a, c, g, or t
tncarytngtng